(19) United States
(12) Patent Application Publication
(10) Pub. No.: US 2024/0132938 A1
(43) Pub. Date: Apr. 25, 2024

(54) REDUCING SPATIAL OCCUPANCY OF MOLECULES IN A SAMPLE THROUGH SAMPLE CROSSLINKING

(71) Applicant: 10x Genomics, Inc., Pleasanton, CA (US)

(72) Inventor: Malte KÜHNEMUND, Stockholm (SE)

(21) Appl. No.: 18/478,392

(22) Filed: Sep. 29, 2023

Related U.S. Application Data
(60) Provisional application No. 63/412,375, filed on Oct. 1, 2022.

(51) Int. Cl. C12Q 1/6816; C12Q 1/6806; C12Q 1/6844; C12Q 1/6876

(52) U.S. Cl. CPC C12Q 1/6816; C12Q 1/6806; C12Q 1/6844; C12Q 1/6876

(57) ABSTRACT

The present disclosure in some aspects relates to methods and compositions for confining molecules generated in a biological sample. In particular examples, rolling circle amplification (RCA) products are generated in a fixed biological sample which has been crosslinked prior to RCA, such that the three-dimensional crosslinked matrix generated prior to RCA confines the RCA products, and the RCA products are smaller than they would otherwise be in a fixed biological sample which has not been crosslinked prior to RCA. Confining the RCA products by generating them in a pre-crosslinked matrix in the biological sample may result in compaction of the RCA products and facilitate subsequent in situ analysis. The sample crosslinking can be performed after probe hybridization to nucleic acid molecules in the sample and before RCA.

### CROSS-REFERENCE TO RELATED APPLICATION

This application claims priority to U.S. Provisional Patent Application No. 63/412,375, filed Oct. 1, 2022, entitled “REDUCING SPATIAL OCCUPANCY OF MOLECULES IN A SAMPLE THROUGH SAMPLE CROSSLINKING,” which is herein incorporated by reference in its entirety for all purposes.

### FIELD

The present disclosure relates in some aspects to methods and compositions for processing a biological sample, such as methods and compositions for confining molecules in a three-dimensional matrix in a biological sample for in situ analysis.

### BACKGROUND

Methods are available for processing nucleic acids in a biological sample in situ, such as a cell or a tissue. For instance, advances in fluorescent in situ sequencing (FISSEQ) have enabled genome-wide profiling of gene expression in situ in fixed cells and tissues in a three-dimensional matrix. However, these techniques require incorporating aminoallyl-modified dUTPs during nucleic acid amplification and subsequent crosslinking, to achieve stabilization and/or compaction of the nucleic acid molecules. Improved methods for sample processing for in situ analyte detection are needed. The present disclosure addresses these and other needs.

### BRIEF SUMMARY

There is a need for confining structures comprising nucleic acid molecules (e.g., nucleic acid concatemers, such as RCA products) during in situ analysis of a biological sample (e.g., a cell or tissue sample). For example, restricting nucleic acid molecules in the sample may allow for better resolution of signals and resolution of individual nucleic acid molecules. Furthermore, physically limiting the space the is occupied by structures comprising nucleic acids may prevent or reduce the expansion of the structures, resulting in compaction and/or stabilization of the structures for improved spatial fidelity and detection during downstream analyses.

In some embodiments, provided herein are methods and compositions for nucleic acid molecule detection in a biological sample having a three-dimensional matrix that can confine the space occupancy of the nucleic acid molecule, e.g., as it is being generated in the biological sample. In some embodiments, a three-dimensional matrix may reduce the physical space that a nucleic acid concatemer occupies in a biological sample compared to a nucleic acid concatemer that is not in a crosslinked matrix, thereby reducing the diameter of the nucleic acid concatemer and create a greater distance between adjacent nucleic acid concatemers in the biological sample. In some embodiments, the nucleic acid concatemer as it is being generated (e.g., in an amplification reaction) in the crosslinked matrix is physically limited and minimally expands in space, thus confining the nucleic acid concatemer. In some cases, the nucleic acid concatemer as it is being generated in the crosslinked matrix forms certain structures in space (e.g., stacks). In particular, the present disclosures provides methods for confining structures comprising nucleic acid molecules, such as nucleic acid concatemers, in situ in a crosslinked matrix in a biological sample without covalently binding the crosslinked matrix to nucleic acid residues incorporated into the structures by a polymerase, including methods for confining of nucleic acid molecules in space for in situ analysis, e.g., for decoding nucleic acid barcode sequences through sequential cycles of detectable probe hybridization (directly or indirectly) to the nucleic acid molecules (e.g., RCA products). The confining may be achieved through contacting the biological sample with a polymerization agent (e.g., a crosslinking agent) that is capable of forming a crosslinked matrix within the biological sample, e.g., prior to generating the nucleic acid concatemers in the sample.

In some embodiments, disclosed herein is a method for sample analysis, comprising: (a) contacting a biological sample with a crosslinking agent to form a crosslinked matrix in the biological sample, wherein the biological sample is fixed prior to contacting the crosslinking agent; and (b) using a polymerase to generate a rolling circle amplification (RCA) product in the crosslinked matrix, wherein the crosslinked matrix is not covalently bound to nucleic acid residues of the RCA product. In some embodiments, the method comprises: (a) contacting a biological sample with a crosslinking agent to form a crosslinked matrix in the biological sample, wherein the biological sample is fixed prior to contacting the crosslinking agent; and (b) generating a rolling circle amplification (RCA) product in the crosslinked matrix, wherein the crosslinked matrix is not covalently bound to nucleic acid residues incorporated into the RCA product. In some embodiments, the crosslinked matrix confines and/or compacts the RCA product in the crosslinked matrix.

In any of the embodiments herein, the crosslinking agent can be a non-nucleic acid molecule. In any of the embodiments herein, the crosslinking agent can comprise a functional group A that reacts with a functional group B in molecules in the biological sample to form a covalent bond. In some embodiments, the functional group A in the crosslinking agent reacts with the functional group B in molecules that are endogenous in the biological sample. In some embodiments, the endogenous molecules comprise an endogenous nucleic acid, an endogenous protein, an endogenous peptide, an endogenous lipid, an endogenous carbohydrate, and/or an endogenous mineral. In some embodiments, the endogenous molecules comprise extracellular matrix (ECM) components.

In any of the embodiments herein, the crosslinking agent can comprise a plurality of functional groups A that are amine-reactive. In some embodiments, the functional group A is reactive with an α- and/or ε-amino group and not reactive with an aromatic amino group. In some embodiments, the functional group A is reactive with α- and/or ε-amino groups. In any of the embodiments herein, the crosslinking agent can comprise an amine-to-amine crosslinking moiety. In any of the embodiments herein, the crosslinking agent can comprise a bis(sulfosuccinimidyl)suberate (BS3) moiety or a disuccinimidyl suberate (DSS) moiety. In any of the embodiments herein, the crosslinking agent can comprise one or more spacers between functional groups. In some embodiments, the one or more spacers comprise PEG.

In any of the embodiments herein, the crosslinked matrix can comprise molecules of the crosslinking agent covalently bound to themselves. In any of the embodiments herein, the crosslinked matrix can comprise molecules of the crosslinking agent covalently bound to endogenous molecules in the biological sample.

In any of the embodiments herein, the crosslinked matrix may but does not need to be covalently bound to an exogenous molecule or a product or complex thereof prior to generating the RCA product. In some embodiments, the crosslinking agent crosslinks to endogenous biomolecules in tissues, and not to introduced/exogenous/artificial agents introduced into the tissue. In some embodiments, the crosslinked matrix comprises units of one or more crosslinking agents crosslinked to one another and/or to exogenous molecules in the sample. In some embodiments, the crosslinked matrix is not covalently bound to the exogenous molecule or product or complex thereof. In some embodiments, the crosslinked matrix is not covalently bound to an exogenous primer or a product or complex thereof, and the product or complex of the exogenous primer comprises a primer extension product. In some embodiments, the crosslinked matrix is not covalently bound to the product or complex of the exogenous primer, which comprises a cDNA sequence reverse transcribed from an endogenous RNA sequence in the biological sample. In some embodiments, the crosslinked matrix is not covalently bound to an exogenous probe that directly or indirectly binds to an endogenous analyte in the biological sample prior to generating the RCA product (e.g., using the exogenous probe as an RCA template or primer) in the biological sample. In some embodiments, the exogenous molecule is an exogenous probe that directly or indirectly binds to an endogenous analyte in the biological sample. In some embodiments, the exogenous probe comprises a nucleic acid sequence that directly or indirectly binds to an endogenous nucleic acid analyte or a product thereof in the biological sample. In some embodiments, the exogenous probe further comprises a nucleic acid sequence that does not bind to the endogenous nucleic acid analyte or product thereof. In some embodiments, the exogenous probe is a linear, circular, or circularizable probe upon direct or indirect binding to the endogenous nucleic acid analyte or product thereof. In some embodiments, the exogenous probe comprises a binding moiety that directly or indirectly binds to an endogenous non-nucleic acid analyte in the biological sample. In some embodiments, the binding moiety is an antibody or epitope binding fragment thereof. In some embodiments, the non-nucleic acid analyte is a polypeptide. In some embodiments, the exogenous probe further comprises a reporter oligonucleotide that does not bind to the endogenous non-nucleic acid analyte. The reporter oligonucleotide may comprise a barcode sequence that can be detected to identify the binding moiety and/or the corresponding analyte.

In any of the embodiments herein, the biological sample can be contacted with the exogenous probe prior to generating the RCA product. In any of the embodiments herein, the biological sample can be contacted with the exogenous probe prior to the contacting with the crosslinking agent. In any of the embodiments herein, the exogenous probe can be circularized prior to the contacting with the crosslinking agent.

In any of the embodiments herein, the biological sample may but does not need to be subjected to clearing prior to the generating the RCA product. In some embodiments, the biological sample is not subjected to clearing prior to the generating the RCA product. In some embodiments, the clearing comprises hydrophobic clearing, hydrophilic clearing, and/or hydrogel-based clearing. In any of the embodiments herein, the biological sample may but does not need to be subjected to lipid digestion and/or extraction prior to the generating the RCA product. In any of the embodiments herein, the biological sample may but does not need to be subjected to protein digestion and/or extraction prior to the generating the RCA product.

In any of the embodiments herein, the RCA product can be generated at a location in the biological sample. In any of the embodiments herein, the RCA product can be generated from a circular or circularized DNA, a circular or circularized RNA, or a circular or circularized nucleic acid probe or probe set in the biological sample. In any of the embodiments herein, the RCA product can be generated from a circularized cDNA. In any of the embodiments herein, the RCA product can be generated from a circular or circularizable nucleic acid probe or probe set hybridized to a nucleic acid in the biological sample. In some embodiments, the nucleic acid is a genomic DNA, an RNA, a cDNA, or an exogenous nucleic acid probe. In any of the embodiments herein, the circularizable nucleic acid probe or probe set can comprise DNA bases and optionally one or more RNA bases at or near a ligation site in the circularizable nucleic acid probe or probe set. In some embodiments, the one or more RNA bases are at a 3′ ligatable end or within five nucleotide residues from the 3′ ligatable end in the circularizable nucleic acid probe or probe set. In any of the embodiments herein, the nucleic acid can be an mRNA and the circularizable nucleic acid probe or probe set can be a DNA/RNA chimera comprising primarily DNA bases and one or more RNA bases at or within five residues from a 3′ ligatable end.

In any of the embodiments herein, the RCA product can be generated using a circular template that is covalently or noncovalently linked to an endogenous molecule in the biological sample and/or the crosslinked matrix. In some embodiments, the circular template is not covalently linked to the endogenous molecule via the crosslinking agent or the crosslinked matrix.

In any of the embodiments herein, the RCA product can be generated using a circular template that hybridizes to a nucleic acid which is covalently or noncovalently linked to an endogenous molecule in the biological sample and/or the crosslinked matrix. In some embodiments, the nucleic acid is not covalently linked to the endogenous molecule via the crosslinking agent or the crosslinked matrix.

In any of the embodiments herein, the RCA product can be generated using a primer that is covalently or noncovalently linked to endogenous molecules in the biological sample and/or the crosslinked matrix. In some embodiments, the primer is not covalently linked to the endogenous molecule via the crosslinking agent or the crosslinked matrix. In any of the embodiments herein, the biological sample can be contacted with the primer prior to the contacting with the crosslinking agent.

In any of the embodiments herein, the RCA product can be generated in the absence of dNTPs comprising the functional group B. In some embodiments, the RCA product does not comprise nucleic acid residues comprising the functional group B. In some embodiments, the RCA product does not comprise modified nucleic acid residues. In any of the embodiments herein, tandem repeats in the RCA product can be free of nucleic acid residues comprising the functional group B. In any of the embodiments herein, tandem repeats in the RCA product can be free of modified nucleic acid residues. In some embodiments, In some embodiments, the RCA product does not comprise amine-modified nucleic acid residues. In any of the embodiments herein, In some embodiments, the RCA product does not comprise aminoallyl nucleotide residues. In any of the embodiments herein, tandem repeats in the RCA product can be composed entirely of naturally occurring nucleic acid residues. In any of the embodiments herein, the RCA product can be composed entirely of naturally occurring nucleic acid residues.

In any of the embodiments herein, the crosslinked matrix can be charge neutral, positively charged, or negatively charged. In some embodiments, the crosslinked matrix is charge neutral. In some embodiments, the crosslinked matrix is positively charged. In some embodiments, the crosslinked matrix is negatively charged.

In any of the embodiments herein, the diameter of the RCA product can be smaller than the diameter of a reference RCA product generated in a reference biological sample without crosslinking the reference biological sample prior to RCA. In any of the embodiments herein, the crosslinked matrix can confine the RCA product in the biological sample, and the diameter of the confined RCA product is smaller than that of a reference RCA product generated in a reference biological sample without crosslinking the reference biological sample prior to RCA.

In any of the embodiments herein, a plurality of RCA products can be generated in the biological sample, and the average diameter of the plurality of RCA products is smaller than the average diameter of reference RCA products generated in the reference biological sample without crosslinking the reference biological sample prior to RCA. In any of the embodiments herein, a plurality of RCA products can be confined in the biological sample by the crosslinked matrix. In any of the embodiments herein, the average diameter of the confined RCA products can be smaller than that of reference RCA products generated in the reference biological sample without crosslinking the reference biological sample prior to RCA. In any of the embodiments herein, the average diameter of the confined RCA products can be at least about 5% smaller, at least about 10% smaller, at least about 15% smaller, at least about 20% smaller, at least about 25% smaller, or more than about 25% smaller than that of the reference RCA products generated in the reference biological sample. In any of the embodiments herein, the average diameter of the confined RCA products can be less than about 1.5 μm, less than about 1.4 μm, less than about 1.3 μm, less than about 1.2 μm, less than about 1.1 μm, less than about 1 μm, less than about 0.9 μm, less than about 0.8 μm, less than about 0.7 μm, less than about 0.6 μm, less than about 0.5 μm, less than about 0.4 μm, or less than about 0.3 μm. In any of the embodiments herein, the average diameter of the confined RCA products can be about 0.5 μm smaller, about 0.4 μm smaller, about 0.3 μm smaller, about 0.2 μm smaller, about 0.1 μm smaller, or about 0.05 μm smaller than the that of the reference RCA products generated in the reference biological sample. In any of the embodiments herein, the average distance between the confined RCA products in the biological sample can be greater than that of the reference RCA products generated in the reference biological sample.

In any of the embodiments herein, the average length of the plurality of RCA products can be more than about 1 kb, more than about 5 kb, more than about 10 kb, more than about 25 kb, or more than about 50 kb. In any of the embodiments herein, the RCA is performed for about 10 minutes, about 30 minutes, about 1 hour, about 2 hours, about 3 hours, about 5 hours, about 10 hours, about 15 hours, about 20 hours, or within a range between any of the aforementioned reaction times.

In any of the embodiments herein, the method can further comprise: (c) contacting the biological sample with a detectable probe that hybridizes to the RCA product. In any of the embodiments herein, the method can further comprise: (d) detecting a signal associated with the detectable probe at a location in the biological sample, thereby detecting the RCA product at the location.

In any of the embodiments herein, the detectable probe can hybridize to an identifier sequence in the RCA product. In some embodiments, the identifier sequence comprises a nucleic acid analyte sequence or complement thereof. In some embodiments, the identifier sequence comprises a barcode sequence or complement thereof. In some embodiments, the barcode sequence corresponds to an analyte (e.g., a nucleic acid analyte or a non-nucleic acid analyte such as a protein) or a portion thereof.

In any of the embodiments herein, the detectable probe can comprise a fluorescent label. In any of the embodiments herein, the detectable probe can comprise a region for binding to a fluorescently labeled probe. In any of the embodiments herein, the detectable probe can be selected from the group consisting of: a detectable probe comprising a 3′ or 5′ overhang upon hybridization to the RCA product; a detectable probe comprising a 3′ overhang and a 5′ overhang upon hybridization to the RCA product; a circular detectable probe; a circularizable detectable probe or probe set; a detectable probe or probe set comprising a split hybridization region configured to hybridize to a splint; and a combination thereof.

In any of the embodiments herein, the signal associated with the detectable probe can be detected by imaging the biological sample. In some embodiments, the imaging comprises fluorescent imaging, e.g., using a fluorescent microscopy method.

In some embodiments, disclosed herein is a method for sample analysis, comprising: (a) contacting a biological sample with a crosslinking agent to form a crosslinked matrix in the biological sample, wherein: the biological sample is fixed prior to contacting the crosslinking agent, and the crosslinking agent comprises a functional group that reacts with one or more endogenous molecules in the biological sample; and (b) using a polymerase to generate a rolling circle amplification (RCA) product in the crosslinked matrix, wherein: the biological sample is not cleared prior to generating the RCA product, and the crosslinked matrix is not covalently bound to nucleic acid residues in the RCA product.

In some embodiments, disclosed herein is a method for sample analysis, comprising: (a) contacting a biological sample with a crosslinking agent to form a crosslinked matrix in the biological sample, wherein: the biological sample is fixed prior to contacting the crosslinking agent, the crosslinking agent comprises a functional group A that reacts with a functional group B in endogenous molecules in the biological sample, and the crosslinking agent does not react with an exogenous molecule or a product or complex thereof in the biological sample; and (b) generating a rolling circle amplification (RCA) product in the crosslinked matrix, wherein: the biological sample is not cleared prior to generating the RCA product, the crosslinked matrix is not covalently bound to nucleic acid residues incorporated into the RCA product. In some embodiments, a plurality of RCA products are confined in spaces in the biological sample by the crosslinked matrix, and the average diameter of the confined RCA products is smaller than that of reference RCA products in a reference biological sample without the crosslinked matrix prior to the RCA.

In any of the embodiments herein, the average diameter of the confined RCA products can be at least about 5% smaller, at least about 10% smaller, at least about 15% smaller, at least about 20% smaller, at least about 25% smaller, or more than about 25% smaller than that of the reference RCA products in the reference biological sample.

In any of the embodiments herein, the average diameter of the confined RCA products can be less than about 1 μm, less than about 0.9 μm, less than about 0.8 μm, less than about 0.7 μm, less than about 0.6 μm, less than about 0.5 μm, less than about 0.4 μm, or less than about 0.3 μm.

In any of the embodiments herein, the average diameter of the confined RCA products can be about 0.5 μm smaller, about 0.4 μm smaller, about 0.3 μm smaller, about 0.2 μm smaller, about 0.1 μm smaller, or about 0.05 μm smaller than the that of the reference RCA products generated in the reference biological sample.

In any of the embodiments herein, the RCA to generate the plurality of RCA products and/or the RCA to generate the reference RCA products can be performed for about 10 minutes, about 30 minutes, about 1 hour, about 2 hours, about 3 hours, about 5 hours, about 10 hours, about 15 hours, about 20 hours, or within a range between any of the aforementioned reaction times.

In any of the embodiments herein, the method can further comprise: (c) contacting the biological sample with a detectable probe that hybridizes to the RCA product; and (d) detecting a signal associated with the detectable probe at a location in the biological sample, thereby detecting the RCA product at the location.

In some embodiments, provided herein is a method for sample analysis, comprising: (a) fixing a biological sample; (b) contacting the biological sample with a circularizable probe or probe set that that directly or indirectly binds to an analyte in the biological sample; (c) circularizing the circularizable probe or probe set to form a circularized probe; (d) contacting the biological sample with a crosslinking agent to form a crosslinked matrix in the biological sample; (e) generating a rolling circle amplification (RCA) product in the crosslinked matrix using the circularized probe as a template; (f) contacting the biological sample with a detectable probe that hybridizes to the RCA product; and (g) detecting a signal associated with the detectable probe at a location in the biological sample, thereby detecting the RCA product at the location. In some embodiments, the crosslinking agent comprises a functional group A that reacts with a functional group B in endogenous molecules in the biological sample.

In some embodiments, provided herein is a method for sample analysis, comprising: (a) providing a biological sample comprising an analyte, wherein the biological sample is a fixed biological sample; (b) contacting the biological sample with a circularizable probe or probe set that directly or indirectly binds to the analyte in the biological sample; (c) circularizing the circularizable probe or probe set to form a circularized probe; (d) contacting the biological sample with a crosslinking agent to form a crosslinked matrix in the biological sample, wherein the crosslinking agent comprises a functional group that reacts with an endogenous molecule in the biological sample; (e) using a polymerase to generate a rolling circle amplification (RCA) product in the crosslinked matrix using the circularized probe as a template; (f) contacting the biological sample with a detectable probe that hybridizes to the RCA product; and (g) detecting a signal associated with the detectable probe at a location in the biological sample, thereby detecting the RCA product at the location.

In some embodiments, the crosslinking agent does not react with the circularizable probe or probe set or a product or complex thereof in the biological sample. In some embodiments, the crosslinking agent does not react with exogenous probes or products of endogenous molecules in the biological sample. In some embodiments, the endogenous molecules are mRNA and the products are cDNA. In some embodiments, the biological sample is not cleared prior to generating the RCA product. In some embodiments, the crosslinked matrix is not covalently bound to nucleic acid residues incorporated into the RCA product.

In some embodiments, provided herein is a method for sample analysis, comprising: (a) providing a biological sample comprising an analyte, wherein the biological sample is a fixed biological sample; (b) contacting the biological sample with a crosslinking agent to form a crosslinked matrix in the biological sample; (c) contacting the biological sample with a circularizable probe or probe set that directly or indirectly binds to the analyte in the biological sample; (d) circularizing the circularizable probe or probe set to form a circularized probe; (e) using a polymerase to generate a rolling circle amplification (RCA) product in the biological sample using the circularized probe as a template, wherein the polymerase is optionally a phi29 polymerase; (f) contacting the biological sample with a detectable probe that hybridizes to the RCA product; and (g) detecting a signal associated with the detectable probe at a location in the biological sample, thereby detecting the RCA product at the location, thereby detecting the analyte in the fixed biological sample. In some embodiments, the crosslinking agent comprises a functional group that reacts with an endogenous molecule in the biological sample. In some embodiments, the crosslinking agent does not react with the circularizable probe or probe set in the biological sample. In some embodiments, the biological sample is not cleared prior to generating the RCA product. In some embodiments, the crosslinked matrix is not covalently bound to nucleic acid residues in the RCA product.

In some embodiments, provided herein is a method for sample analysis, comprising: (a) fixing a biological sample; (b) contacting the biological sample with a crosslinking agent to form a crosslinked matrix in the biological sample; (c) contacting the biological sample with a circularizable probe or probe set that directly or indirectly binds to an analyte in the biological sample; (d) circularizing the circularizable probe or probe set to form a circularized probe; (e) using a polymerase to generate a rolling circle amplification (RCA) product in the crosslinked matrix using the circularized probe as a template, wherein the polymerase is optionally a phi29 polymerase; (f) contacting the biological sample with a detectable probe that hybridizes to the RCA product; and (g) detecting a signal associated with the detectable probe at a location in the biological sample, thereby detecting the RCA product at the location. In some embodiments, the crosslinking agent comprises a functional group that reacts with an endogenous molecule in the biological sample.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows an exemplary workflow of the methods described herein.

FIG. 2 shows size reduction of rolling circle amplification (RCA) products in a biological sample by a crosslinked matrix formed prior to RCA, thereby compacting the resulting RCA product.

FIGS. 3A-3B show RCA product sizes were reduced in samples crosslinked by BS3 after RCA in the absence of aminoallyl nucleotides, in the dentate gyrus (DG) (FIG. 3A) and in the cortex (CX) (FIG. 3B), compared to PBS control samples.

FIGS. 4A-4B show RCA product counts per nuclei area in samples crosslinked by BS3 after RCA in the absence of aminoallyl nucleotides, in DG (FIG. 4A) and in CX (FIG. 4B), compared to PBS control samples.

FIGS. 5A-5B show RCA product signal intensities above local background in samples crosslinked by BS3 after RCA in the absence of aminoallyl nucleotides, in DG (FIG. 5A) and in CX (FIG. 5B), compared to PBS control samples.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

When utilizing in situ analysis, such as rolling circle amplification (RCA), to detect nucleic acid targets, properly resolving densely packed nucleic acid concatemers, such as rolling circle amplification products (RCPs), in a cell or surface depends upon resolution of the individual nucleic acid concatemers. RCPs are nucleic acid concatemers comprising multiple copies of the target sequence and the RCA probe sequence, which can be visualized using in situ detection probes. Nucleic acid concatemer detection may be enhanced by confining nucleic acid concatemers in the biological sample and/or the crosslinked matrix (e.g., using a three-dimensional matrix) to prevent the flexibility and expansion of the concatemers, as reduction in size results in local concentration of the detection probes to increase signal intensity and resolution of individual concatemers. Confining the nucleic acid concatemer may also allow for stabilization.

Typical strategies for nucleic acid concatemer size reduction using three-dimensional matrices rely on covalently binding the matrix to nucleic acid residues incorporated into the nucleic acid concatemer by a polymerase. The nucleic acid residues are often amine-modified, such as comprising aminoallyl-dU residues, resulting in cross-linking within the RCP and between the RCP and the three-dimensional matrix. However, some sample processing techniques are not compatible with modified nucleic acid residues. Thus, there is a need for methods and compositions for confining nucleic acid concatemers in a three-dimensional matrix without covalently binding the nucleic acid molecules to the matrix or one another. According to some embodiments in the present application, the biological sample may be contacted with a polymerization agent comprising a functional group that specifically reacts with a complementary functional group in a molecule or product thereof in the biological sample, resulting in a crosslinked matrix. The crosslinked matrix does not need to covalently bind to nucleic acid residues incorporated into the nucleic acid concatemer by a polymerase (e.g., RCA primer) to confine the nucleic acid concatemer in the biological sample and/or the crosslinked matrix. Functional groups can be biorthogonal click functional groups, but do not need to be click functional groups, as long as they are able to specifically react with each other to result in the formation of a crosslinked matrix within the biological sample without covalently binding to the nucleic acid concatemer itself. The tight crosslinked matrix formed by the polymerization agent would lead to a reduced physical space, thereby making the nucleic acid concatemers less flexible. Moreover, during washing between rounds of in situ hybridization and target detection, the confined nucleic acid concatemers may have enhanced resistance to swelling and remain inside a confined space within the crosslinked matrix, thereby leading to compaction of the nucleic acid concatemers. In some aspects, additional polymerization agents, polymers, and/or modifications may be added during the formation of the crosslinked matrix. Such agents could generate a more positively charged crosslinked matrix, resulting in nucleic acid concatemers sticking strongly to the crosslinked matrix. Alternatively, these agents could generate a more negatively charged crosslinked matrix, resulting in nucleic acid concatemer repulsion by the crosslinked matrix, thereby stacking on one another.

Provided herein is a method for processing a biological sample, comprising contacting the biological sample with a polymerization agent, wherein the biological sample comprises a nucleic acid concatemer; wherein the polymerization agent comprises a functional group A capable of reacting with a functional group B in a molecule or product thereof in the biological sample to form a crosslinked matrix; wherein the crosslinked matrix is not covalently bound to nucleic acid residues incorporated into the nucleic acid concatemer by a polymerase.

In some aspects, provided herein is a method for processing a biological sample, comprising: a) contacting the biological sample with a crosslinking agent to form a crosslinked matrix in the biological sample, wherein the crosslinking agent comprises a functional group A capable of reacting with a functional group B in endogenous molecules in the biological sample; and b) generating a rolling circle amplification (RCA) product in the biological sample using a polymerase, wherein the crosslinked matrix is not covalently bound to nucleic acid residues incorporated into the RCA product by the polymerase. In some embodiments, the crosslinked matrix confines the RCA product. In some embodiments, the RCA product is confined in a space in the biological sample and/or the crosslinked matrix. In some embodiments, the confined RCA product has a smaller diameter than that of a reference RCA product generated in a reference biological sample without crosslinking the reference biological sample prior to RCA, where the reference biological sample is comparable to the biological sample except that the former is not additionally crosslinked (e.g., in addition to being fixed at the beginning of sample processing) prior to the RCA, and where the confined RCA product and the reference RCA product are generated under identical or similar conditions, such as using the same polymerase and identical or similar reaction temperatures, reaction time, and/or reagent concentrations, etc. For instance, the reference biological sample and the biological sample can be consecutive tissue sections from the same tissue block.

In some aspects, provided herein is a method for processing a biological sample, comprising: a) generating a rolling circle amplification (RCA) product in the biological sample using a polymerase; and b) contacting the biological sample with a crosslinking agent to form a crosslinked matrix in the biological sample; wherein the crosslinking agent comprises a functional group A capable of reacting with a functional group B in endogenous molecules in the biological sample; wherein the crosslinked matrix is not covalently bound to nucleic acid residues incorporated into the RCA product by the polymerase. In some aspects, the crosslinked matrix confines the RCA product in the biological sample and/or the crosslinked matrix. In some aspects, the crosslinked matrix confines the RCA product in a space the biological sample and/or the crosslinked matrix. In some embodiments, the confined RCA product has a smaller diameter than that of a reference RCA product generated in a reference biological sample without crosslinking the reference biological sample prior to RCA.

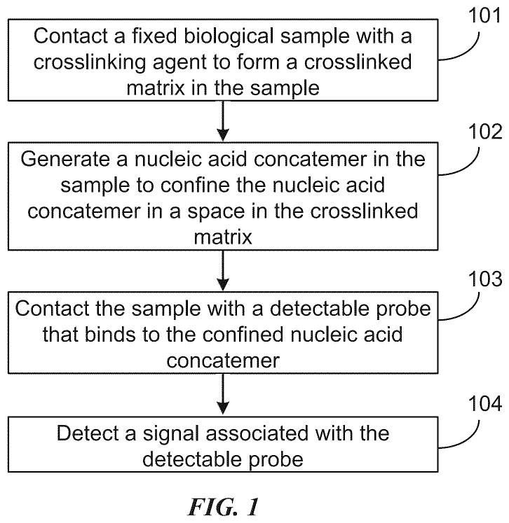

FIG. 1 shows an exemplary workflow for confining a biomolecule in situ in a biological sample, e.g., a fixed biological sample. The biomolecule may be a nucleic acid concatemer, such as an RCA product. In some embodiments, the biological sample is contacted with a polymerization agent (e.g., a crosslinking agent) (e.g., 101 in FIG. 1). Step 101 in FIG. 1 can be performed after hybridization of a circular probe or circularizable probe or probe set, and/or after the ligation of circularizable probe or probe set, and/or after RCA primer hybridization to the circular or circularized probe, but before the RCA reaction. In some embodiments, the nucleic acid concatemer is generated in the biological sample (e.g., 102 in FIG. 1). The polymerized agent may form a crosslinked matrix. The nucleic acid concatemer may be generated in the biological sample prior, to, during, and/or after contacting the biological sample with a polymerization agent. In some embodiments, the nucleic acid concatemer is generated after forming a crosslinked matrix in the biological sample, e.g., using a crosslinking agent that forms covalent bonds with one another and/or with endogenous molecules in the biological sample. In some embodiments, the crosslinked matrix is not covalently cross-linked to the biomolecule (e.g., the nucleic acid concatemer). In some embodiments, contacting the biological sample with the polymerization agent confines the biomolecule in the biological sample and/or the crosslinked matrix. The biomolecule may be detected in the biological sample (e.g., 103 in FIG. 1). Confining the biomolecule in the crosslinked matrix in the biological sample may allow for increased resolution during detection of optical signals associated with analytes in the sample (e.g., 104 in FIG. 1).

Overall, described herein are improved methods for processing a biological sample comprising a biomolecule, which may have applications for in situ analysis of biomolecules, such as nucleic acid concatemers.

In the following sections, additional description of various aspects of the methods, compositions, and kits disclosed herein is provided. Section II describes methods for confining nucleic acid concatemers in the biological sample and/or the crosslinked matrix using a crosslinked matrix; Section III describes exemplary methods for detection and analysis of the biomolecules; and Section IV describes exemplary biological samples as well as analytes (e.g., endogenous analytes, labeling agents, or products of endogenous nucleic analytes) that can be detected using a method of analyzing a biological sample described herein (e.g., by detecting a target nucleic acid that is or is associated with the analyte). Section V provides kits according to the present disclosure, and Section VI describes exemplary applications of the present methods, compositions, and kits. As stated above, the section headings used herein are for organizational purposes only and are not to be construed as limiting the subject matter described.

### II. Confining Nucleic Acid Concatemers in a Biological Sample

This application provides, inter alia, a method for confining a nucleic acid concatemer in a biological sample using a polymerization agent. In some embodiments, the nucleic acid concatemer is confined in a space in the biological sample. In some embodiments, provided herein are methods for reducing the physical space that a nucleic acid concatemer occupies in a biological sample using a polymerization agent. In some embodiments, the diameter of the nucleic acid concatemer is reduced in the biological sample compared to a nucleic acid concatemer in a reference biological sample without using a polymerization agent in the reference biological sample. In some embodiments, the distance between nucleic acid concatemers in the biological sample is increased compared to a nucleic acid concatemer in a reference biological sample without using a polymerization agent in the reference biological sample. In some aspects, provided herein is a method for confining a nucleic acid concatemer via a crosslinked matrix formed using a polymerization agent, wherein the crosslinked matrix is not covalently bound to nucleic acid residues incorporated into the nucleic acid concatemer by a polymerase. In yet other aspects, provided herein is a method for compaction of a nucleic acid concatemer by confining the nucleic acid concatemer in a biological sample using a polymerization agent. In some embodiments, the biological sample is fixed and/or crosslinked prior to contacting with the polymerization agent, and polymerization agent is used to provide additional crosslinking of the fixed and/or crosslinked sample prior to generating nucleic acid products (e.g., nucleic acid concatemers, such as RCPs generated using RCA) in the biological sample. In some embodiments, the additional crosslinking of the biological sample prior to RCA reduces spatial occupancy and the size of RCPs subsequently generated in the biological sample. In some embodiments, the biological sample crosslinked using the polymerization agent prior to RCA confines the RCPs, thereby reducing the average size of the RCPs compared to those generated in a biological sample that has not been crosslinked prior to RCA, and the reduction in RCP size does not depend on crosslinking an RCP to itself and/or to other molecules in the sample.

In some embodiments, a method disclosed herein comprises: fixing a biological sample (e.g., using PFA or other fixatives such as a crosslinking fixative and/or a non-crosslinking fixative); crosslinking the fixed biological sample using a polymerization agent; and performing RCA in the fixed biological sample that has been crosslinked. In some embodiments, a method disclosed herein comprises: fixing a biological sample (e.g., using PFA or other fixatives such as a crosslinking fixative and/or a non-crosslinking fixative); crosslinking the fixed biological sample using a polymerization agent prior to RCA; performing RCA in the fixed biological sample that has been crosslinked; embedding the biological sample comprising RCPs in a matrix such as a hydrogel, where an RCP may be crosslinked to itself, the matrix, and/or other molecules in the sample, and as an example, the RCP crosslinking can be via modified (e.g., amine-modified) nucleotides that have been incorporated into the RCP during RCA. In some embodiments, a method disclosed herein comprises: fixing a biological sample (e.g., using PFA or other fixatives such as a crosslinking fixative and/or a non-crosslinking fixative); embedding the biological sample (which does not comprise RCPs) in a matrix such as a hydrogel, and optionally clearing the embedded biological sample; crosslinking the embedded and optionally cleared biological sample using a polymerization agent prior to RCA; performing RCA in the sample that has been crosslinked, where RCPs may be crosslinked to itself, the matrix, or other molecules in the sample, and the RCP crosslinking can be via modified (e.g., click-modified) nucleotides that have been incorporated into the RCP during RCA. In some embodiments, a method disclosed herein comprises: fixing a biological sample (e.g., using PFA or other fixatives such as a crosslinking fixative and/or a non-crosslinking fixative); crosslinking cDNA to the cell matrix in the biological sample (which does not comprise RCPs), such as using BS(PEG)₉ to crosslink primary amines introduced into the cDNA during reverse transcription); crosslinking the biological sample using a polymerization agent prior to RCA; performing RCA in the sample that has been crosslinked using the polymerization agent, where RCPs may be crosslinked to itself, a matrix, or other molecules in the sample, and the RCP crosslinking can be via modified (e.g., amine-modified) nucleotides that have been incorporated into the RCP during RCA. In some embodiments, the diameters of RCPs are measurable (e.g., visualized under a microscope) as smaller when compacted by the crosslinked matrix in the biological sample crosslinked before RCA, as compared to the diameters of RCPs generated without sample crosslinking before RCA. In some embodiments, the distances between adjacent RCPs are larger when the RCPs themselves compacted by the crosslinked matrix in the biological sample crosslinked before RCA, as compared to the distances between adjacent RCPs generated without sample crosslinking before RCA.

In any of the embodiments herein, the sample crosslinking provided by the polymerization agent (including samples that have been fixed and/or crosslinked prior to crosslinking using the polymerization agent) can be performed in the middle of library preparation, such as after circular or circularizable probe hybridization to nucleic acid molecules in the sample and optional probe circularization (e.g., via ligation) and before RCA. In some embodiments, a biological sample (e.g., a fresh frozen sample or a stored or archived sample) can be fixed using a fixative (e.g., PFA, or other fixatives such as methanol (MeOH), or an agent that fixes and permeabilizes the sample) during early sample preparation, followed by crosslinking the sample in the middle of or as a part of library preparation, where the sample can be crosslinked using BS3 or other crosslinker or fixative. In some embodiments, the crosslinking was done after probe ligation and/or primer hybridization. In some embodiments, the primer is crosslinked to the sample (e.g., a tissue) with crosslinker or fixative (e.g., BS3), but the crosslinking also crosslinks the entire sample (e.g., tissue matrix) again.

A. Nucleic Acid Concatemer

In some aspects, provided herein are methods and compositions for processing a biological sample comprising a nucleic acid concatemer, and methods and compositions processing a biological sample comprising a molecule for generating a nucleic acid concatemer from an analyte (e.g., nucleic acid analyte) in the biological sample.

Provided herein is a method for sample analysis, comprising contacting a biological sample with a crosslinking agent to form a crosslinked matrix in the biological sample, wherein the biological sample is fixed prior to contacting the crosslinking agent, then generating a rolling circle amplification (RCA) product in the crosslinked matrix, wherein the crosslinked matrix is not covalently bound to nucleic acid residues incorporated into the RCA product. In some embodiments, the biological sample is fixed or otherwise treated to preserve the biological structure and components (e.g., nucleic acids) of the sample prior to contacting the sample with the crosslinking agent to form the crosslinked matrix. For example, a sample can be fixed via immersion in ethanol, methanol, acetone, paraformaldehyde (PFA)-Triton, and combinations thereof, e.g., described in Section III.A.(iii). Sample processing by fixation (e.g., using PFA), which usually occurs at the beginning of sample processing, and the crosslinking of the fixed sample are separate steps. For example, the crosslinking can be after probe hybridization and/or ligation in a fixed and permeabilized sample, such that the probes and reagents (e.g., ligases) have better access to molecules in the sample prior to sample crosslinking, whereas after sample crosslinking the crosslinked matrix can confine the physical space occupied by the nucleic acid concatemers (e.g., RCPs) as they are being generated in the crosslinked sample. In some aspects, contacting the sample with the crosslinking agent to form the crosslinked matrix is a separate step performed after the biological sample was preserved in a prior step. In some embodiments, the crosslinking agent is used to confine the spatial occupancy of each of the amplification products generated in the sample.

In some embodiments, a fixed or preserved biological sample is permeabilized prior to being contacted with the crosslinking agent. In some embodiments, after the sample is prepared (e.g., fixed), a hybridization complex comprising a probe and a target is formed in a sample. In some cases, a ligation reaction can be then performed to generate circularizable probes (e.g., for use as template for an RCA reaction). In some cases, after probes are hybridized, the method comprises contacting a biological sample with a crosslinking agent to form a crosslinked matrix in the biological sample. In some cases, after probe ligation and primer hybridization, the method comprises contacting a biological sample with a crosslinking agent to form a crosslinked matrix in the biological sample.

(i) Molecule for Generating a Nucleic Acid Concatemer

In some embodiments, the biological sample comprises one or more molecule(s) for generating a nucleic acid concatemer of an analyte in the biological sample. In some embodiments, the molecule for generating a nucleic acid concatemer is a circular DNA that comprises one or more DNA bases. In some embodiments, the circular DNA is a circularized cDNA. The circular DNA molecules may be amplified to generate nucleic acid concatemers, such as isothermal enzymatic amplification, inducing but not limited to rolling circle amplification (RCA). In some embodiments, the nucleic acid concatemer is in proximity with the circular DNA. In some embodiments, one or more nucleic acid concatemers assemble around the circular DNA. Each circular DNA may have more than 1000 nucleic acid concatemers surrounding or otherwise associated therewith. In some embodiments, the molecule for generating a nucleic acid concatemer is a circular RNA that comprises one or more RNA bases. In some embodiments, the molecule for generating a nucleic acid concatemer is a circular DNA that comprises one or more DNA bases and one or more RNA bases. In some embodiments, molecule for generating a nucleic acid concatemer is a circular RNA that comprises one or more DNA bases and one or more RNA bases.

In some embodiments, the molecule for generating a nucleic acid concatemer (e.g., used as template) is a probe that comprises one or more DNA bases. In some embodiments, the molecule for generating a nucleic acid concatemer is a probe that comprises one or more RNA bases. In some embodiments, the molecule for generating a nucleic acid concatemer is a probe that comprises one or more DNA bases and one or more RNA bases. In some embodiments, the probe is a circular probe. In some embodiments, the circular probe is a circularized probe generated from a circularizable probe or probe set hybridized to a nucleic acid (e.g., a genomic DNA, an mRNA, a cDNA, or a probe) in the biological sample. In some embodiments, the probe may be used to amplify an analyte, such as a nucleic acid molecule analyte (e.g., any of the analytes described in Section III.B, below), in the biological sample to generate nucleic acid concatemers, such as isothermal enzymatic amplification, inducing but not limited to RCA.

(a) Hybridization and Ligation

In some embodiments, the molecule for generating a nucleic acid concatemer (e.g., used as template) is a probe that comprises a sequence substantially complementary or complementary to an endogenous analyte and/or a labeling agent. For example, the probe hybridizes to a sequence of an endogenous analyte (e.g., RNA). In some embodiments, the probe is contacted with the biological sample prior to the sample being crosslinked (e.g., as described in II.B.). In some embodiments, the probe is contacted with the biological sample and ligated prior to the sample being crosslinked (e.g., as described in II.B.).

In some embodiments, the molecule for generating a nucleic acid concatemer is a circular probe or circularizable probe or probe set. For instance, a circularizable probe or probe set disclosed herein can comprise a padlock probe that does require gap filling to circularize upon hybridization to a template (e.g., a target nucleic acid and/or a probe such as a splint), a gapped padlock probe (e.g., one that require gap filling to circularize upon hybridization to a template), an L-shaped probe (e.g., one that comprises a target recognition sequence and a 5′ or 3′ overhang upon hybridization to a target nucleic acid or a probe), a U-shaped probe (e.g., one that comprises a target recognition sequence, a 5′ overhang, and a 3′ overhang upon hybridization to a target nucleic acid or a probe), a V-shaped probe (e.g., one that comprises at least two target recognition sequences and a linker or spacer between the target recognition sequences upon hybridization to a target nucleic acid or a probe), a probe or probe set for proximity ligation (such as those described in U.S. Pat. Nos. 7,914,987 and 8,580,504 incorporated herein by reference in their entireties, and probes for Proximity Ligation Assay (PLA) for the simultaneous detection and quantification of nucleic acid molecules and protein-protein interactions), or any suitable combination thereof. In some embodiments, a circularizable probe or probe set disclosed herein can comprise a probe that is ligated to itself or another probe using DNA-templated and/or RNA-templated ligation. In some embodiments, a circularizable probe or probe set disclosed herein can be a DNA molecule and can comprise one or more other types of nucleotides, modified nucleotides, and/or nucleotide analogues, such as one or more ribonucleotides. In some embodiments, the ligation can be a DNA ligation on a DNA template. In some embodiments, the ligation can be a DNA ligation on an RNA template, and the probes can comprise RNA-templated ligation probes. In some embodiments, a circularizable probe or probe set disclosed herein can comprise a padlock-like probe or probe set. In some embodiments, a nucleic acid probe disclosed herein is part of a SNAIL (Splint Nucleotide Assisted Intramolecular Ligation) probe set, such as one described in US 2019/0055594 or US 2021/0164039 which are incorporated herein by reference in their entireties. In some embodiments, a nucleic acid probe disclosed herein is part of a PLAYR (Proximity Ligation Assay for RNA) probe set, such as one described in US 2016/0108458 which is incorporated herein by reference in its entirety. In some embodiments, a nucleic acid probe disclosed herein is part of a PLISH (Proximity Ligation in situ Hybridization) probe set, such as one described in US 2020/0224243 which is incorporated herein by reference in its entirety. Any suitable combination of the probe designs described herein can be used.

Any suitable circularizable probe or probe set, or indeed more generally circularizable reporter molecules, may be used to generate the RCA template which is used to generate the RCA product. For example, a circularizable probe or probe set may be used to generate a circular nucleic acid comprising a target hybridization region. The target hybridization region in the RCA product can comprise an identifying sequence, such as a sequence of a nucleic acid analyte or probe to which the circularizable probe or probe set hybridizes, or a barcode sequence. The circularizable probe or reporter (the RCA template) can be in the form of a linear molecule having ligatable ends which may be circularized by ligating the ends together directly or indirectly, e.g., to each other, or to the respective ends of an intervening (“gap”) oligonucleotide or to an extended 3′ end of the circularizable RCA template. A circularizable template may also be provided in two or more parts, namely two or more molecules (e.g., oligonucleotides) which may be ligated together to form a circle. When said RCA template is circularizable it is circularized by ligation prior to RCA. Ligation may be templated using a ligation template, and in the case of padlock and molecular inversion probes and such like the target analyte may provide the ligation template, or it may be separately provided. The circularizable RCA template (or template part or portion) will comprise at its respective 3′ and 5′ ends regions of complementarity to corresponding cognate complementary regions (or binding sites) in the ligation template, which may be adjacent where the ends are directly ligated to each other, or non-adjacent, with an intervening “gap” sequence, where indirect ligation is to take place.

In the case of padlock probes, in one embodiment the ends of the padlock probe may be brought into proximity to each other by hybridization to adjacent sequences on a target nucleic acid molecule (such as a target analyte), which acts as a ligation template, thus allowing the ends to be ligated together to form a circular nucleic acid molecule, allowing the circularized padlock probe to act as a template for an RCA reaction. In such an example the terminal sequences of the padlock probe which hybridize to the target nucleic acid molecule will be specific to the target analyte in question, and will be replicated repeatedly in the RCA product. They may therefore act as a marker sequence indicative of that target analyte. Accordingly, it can be seen that the marker sequence in the RCA product may be equivalent to a sequence present in the target analyte itself. Alternatively, a marker sequence (e.g. tag or barcode sequence) may be provided in the non-target complementary parts of the padlock probe. In still a further embodiment, the marker sequence may be present in the gap oligonucleotide which is hybridized between the respective hybridized ends of the padlock probe, where they are hybridized to non-adjacent sequences in the target molecule. Such gap-filling padlock probes are akin to molecular inversion probes.

In some embodiments, similar circular RCA template molecules can be generated using molecular inversion probes. Like padlock probes, these are also typically linear nucleic acid molecules capable of hybridizing to a target nucleic acid molecule (such as a target analyte) and being circularized. The two ends of the molecular inversion probe may hybridize to the target nucleic acid molecule at sites which are proximate but not directly adjacent to each other, resulting in a gap between the two ends. The size of this gap may range from only a single nucleotide in some embodiments, to larger gaps of 100 to 500 nucleotides, or longer, in other embodiments. Accordingly, it is necessary to supply a polymerase and a source of nucleotides, or an additional gap-filling oligonucleotide, in order to fill the gap between the two ends of the molecular inversion probe, such that it can be circularized.

As with the padlock probe, the terminal sequences of the molecular inversion probe which hybridize to the target nucleic acid molecule, and the sequence between them, will be specific to the target analyte in question, and will be replicated repeatedly in the RCA product. They may therefore act as a marker sequence indicative of that target analyte. Alternatively, a marker sequence (e.g., tag or barcode sequence) may be provided in the non-target complementary parts of the molecular inversion probe.

In some embodiments, the probes disclosed herein may be invader probes, e.g., for generating a circular nucleic acid such as a circularized probe. Such probes are of particular utility in the detection of single nucleotide polymorphisms. The detection method of the present disclosure may, therefore, be used in the detection of a single nucleotide polymorphism, or indeed any variant base, in the target nucleic acid sequence. Probes for use in such a method may be designed such that the 3′ ligatable end of the probe is complementary to and capable of hybridizing to the nucleotide in the target molecule which is of interest (the variant nucleotide), and the nucleotide at the 3′ end of the 5′ additional sequence at the 5′ end of the probe or at the 5′ end of another, different, probe part is complementary to the same said nucleotide, but is prevented from hybridizing thereto by a 3′ ligatable end (e.g., it is a displaced nucleotide). Cleavage of the probe to remove the additional sequence provides a 5′ ligatable end, which may be ligated to the 3′ ligatable end of the probe or probe part if the 3′ ligatable end is hybridized correctly to (e.g. is complementary to) the target nucleic acid molecule. Probes designed according to this principle provide a high degree of discrimination between different variants at the position of interest, as only probes in which the 3′ ligatable end is complementary to the nucleotide at the position of interest may participate in a ligation reaction. In one embodiment, the probe is provided in a single part, and the 3′ and 5′ ligatable ends are provided by the same probe. In some embodiments, an invader probe is a padlock probe (an invader padlock or “iLock”), e.g., as described in Krzywkowski et al., Nucleic Acids Research 45, e161, 2017 and US 2020/0224244, which are incorporated herein by reference.

Other types of probes which result in circular molecules which can be detected by RCA and which comprise either a target analyte sequence or a complement thereof include selector-type probes described in US2019/0144940, which comprise sequences capable of directing the cleavage of a target nucleic acid molecule (e.g. a target analyte) so as to release a fragment comprising a target sequence from the target analyte and sequences capable of templating the circularization and ligation of the fragment. WO 2016/016452, which is incorporated herein by reference in its entirety, describes probes which comprise a 3′ sequence capable of hybridizing to a target nucleic acid molecule (e.g. a target analyte) and acting as a primer for the production of a complement of a target sequence within the target nucleic acid molecule (e.g. by target templated extension of the primer), and an internal sequence capable of templating the circularization and ligation of the extended probe comprising the reverse complement of the target sequence within the target analyte and a portion of the probe. In the case of both such probes, target sequences or complements thereof are incorporated into a circularized molecule which acts as the template for the RCA reaction to generate the RCA product, which consequently comprises concatenated repeats of said target sequence. In some embodiments, said target sequence may act as, or may comprise a marker sequence within the RCA product indicative of the target analyte in question. Alternatively, a marker sequence (e.g. tag or barcode sequence) may be provided in the non-target complementary parts of the probes.

In some embodiments, a nucleic acid probe disclosed herein can be pre-assembled from multiple components, e.g., prior to contacting the nucleic acid probe with a target nucleic acid or a sample. In some embodiments, a nucleic acid probe disclosed herein can be assembled during and/or after contacting a target nucleic acid or a sample with multiple components. In some embodiments, a nucleic acid probe disclosed herein is assembled in situ in a sample. In some embodiments, the multiple components can be contacted with a target nucleic acid or a sample in any suitable order and any suitable combination. For instance, a first component and a second component can be contacted with a target nucleic acid, to allow binding between the components and/or binding between the first and/or second components with the target nucleic acid. Optionally a reaction involving either or both components and/or the target nucleic acid, between the components, and/or between either one or both components and the target nucleic acid can be performed, such as hybridization, ligation, primer extension and/or amplification, chemical or enzymatic cleavage, click chemistry, or any combination thereof. In some embodiments, a third component can be added prior to, during, or after the reaction. In some embodiments, a third component can be added prior to, during, or after contacting the sample with the first and/or second components. In some embodiments, the first, second, and third components can be contacted with the sample in any suitable combination, sequentially or simultaneously. In some embodiments, the nucleic acid probe can be assembled in situ in a stepwise manner, each step with the addition of one or more components, or in a dynamic process where all components are assembled together. One or more removing steps, e.g., by washing the sample such as under stringent conditions, may be performed at any point during the assembling process to remove or destabilize undesired intermediates and/or components at that point and increase the chance of accurate probe assembly and specific target binding of the assembled probe.

In some embodiments, the molecule for generating a nucleic acid concatemer (e.g., used as template) is a probe that can be ligated when hybridized to a target analyte (e.g., sequence of an endogenous analyte (e.g., RNA)). In some embodiments, the ligation involves chemical ligation. In some embodiments, the ligation involves template dependent ligation. In some embodiments, the ligation involves template independent ligation. In some embodiments, the ligation involves enzymatic ligation. In some embodiments, ligation is performed to generate a circularized probe that can serve as a template for an amplification reaction to generate a nucleic acid concatemer.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(b) Primer Extension and Amplification

In some embodiments, probes may be amplified to generate nucleic acid concatemers, such as isothermal enzymatic amplification, inducing but not limited to rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof. In some embodiments, the primer for amplification can be crosslinked to a crosslinked matrix described in II.B.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, the amplification (e.g., RCA) is performed for about 10 minutes, about 30 minutes, about 1 hour, about 2 hours, about 3 hours, about 5 hours, about 10 hours, about 15 hours, about 20 hours, or within a range between any of the aforementioned reaction times. In some embodiments, the RCA is performed for about 10 minutes, about 20 minutes, about 30 minutes, about 40 minutes, about 50 minutes or about 60 minutes. In any of the embodiments herein, the RCA can be performed for about 30 minutes, about 1 hour, about 2 hours, about 3 hours, about 6 hours, or within a range between any of the aforementioned reaction times.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) can include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:e118, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification. In some cases, the crosslinking of modified nucleotides in the amplification products provides an additional feature in addition to the use of the polymerization agents described herein.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, U.S. Pat. Nos. 10,138,509, 10,266,888, US 2016/0024555, US 2018/0251833 and US 2017/0219465, which are incorporated herein by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be contained within the matrix generally at the location of the nucleic acid being amplified, thereby creating a localized colony of amplicons. The amplification products may be contained within the matrix by steric factors. The amplification products may be contained within the matrix by positive or negative charges. In some cases, the amplification products may also be contained within the matrix by covalent or noncovalent bonding. By being contained by the matrix, the size and spatial relationship of the original amplicons is maintained. In some aspects, the amplification products are resistant to movement or unraveling under mechanical stress. In some embodiments, amplification products may be additionally attached to the matrix generally at the location of the nucleic acid being amplified (e.g., via modified nucleotides).

In some aspects, the amplification products are copolymerized and/or contained by the surrounding matrix thereby preserving their spatial relationship and any information inherent thereto. For example, if the amplification products are those generated from DNA or RNA within a cell embedded in the matrix, the amplification products can also be contained in the matrix preserving their spatial information within the cell thereby providing a subcellular localization distribution pattern. In some embodiments, amplification products additionally attached to the matrix (e.g., via modified nucleotides) preserves their spatial information.

In some embodiments, the RCA template may comprise the target analyte, or a part thereof, where the target analyte is a nucleic acid, or it may be provided or generated as a proxy, or a marker, for the analyte. In some embodiments, assays for the detection of numerous different analytes can use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g., circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

(ii) Nucleic Acid Concatemer

In some embodiments, the biological sample comprises a nucleic acid concatemer, such as a product (e.g., an amplification product, such as a RCP) of an analyte in the biological sample. In some embodiments, the nucleic acid concatemer is an amplification product generated in a crosslinked matrix. In some embodiments, the nucleic acid concatemer is a product of a labeling agent that directly or indirectly binds to an analyte in the biological sample. In some embodiments, the analyte is a nucleic acid analyte or a non-nucleic acid analyte, and the labeling agent comprises a reporter oligonucleotide and an analyte-binding moiety coupled thereto. In some embodiments, the nucleic acid concatemer (e.g., RCP) comprises nucleic acids. In some embodiments, the nucleic acid concatemer contains natural and unnatural nucleotides. For example, the nucleic acid concatemer may comprise modified nucleotides, non-nucleotides, or synthetic nucleotides. Nucleotides amendable to the present application include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Additional suitable bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages. In some embodiments, the modified nucleotides are amine-modified nucleotides.

In some embodiments, the nucleic acid concatemer is between about 1 and about 85 kilobases in length, such as between any of about 1 and about 15 kilobases, about 10 and about 30 kilobases, about 20 and about 40 kilobases, about 30 and about 50 kilobases, about 40 and about 60 kilobases, about 50 and about 70 kilobases, and about 60 and about 85 kilobases in length. In some embodiments, the nucleic acid concatemer is at least 1 kilobase in length, such as any of about 15, 25, 35, 45, 55, 65, or 85 kilobases in length. In some embodiments, the nucleic acid concatemer is more than 85 kilobases in length.

In some embodiments, the nucleic acid concatemer is a rolling circle amplification product comprising a barcode sequence corresponding to an analyte in the biological sample, and the nucleic acid concatemer is between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length.

In some embodiments, the nucleic acid concatemer forms a nanoball such as one having a diameter between about 0.1 μm and about 3 μm. The nanoball may be a product of amplification, such as RCA. In some embodiments, the nanoball has a diameter of between about 0.1 μm and about 4 μm, such as between any of about 0.1 μm and about 0.5 μm, about 0.2 μm and about 2 μm, about 1 μm and about 3 μm, and about 2 μm and about 4 μm. In some embodiments, the nanoball has a diameter of between about 0.2 μm and about 0.8 μm. In some embodiments, the nanoball diameter is at least about 0.1 μm, such as at least any of about 0.2 μm, 0.5 μm, 1 μm, 1.5 μm, 2 μm, 2.5 μm, 3 μm, 3.5 μm, and 4 μm.

In some embodiments, a nucleic acid concatemer is generated from a nucleic acid analyte. For example, the nucleic acid analyte may be DNA, ssDNA, or RNA. In some embodiments, the analyte is a non-nucleic acid analyte (e.g., a protein). In some embodiments, the nucleic acid concatemer is generated from a labeling agent that directly or indirectly binds to an analyte in the biological sample. Examples of labeling agents are described in Section III.B.ii

In some embodiments, the nucleic acid concatemer is an amplification product, such as a rolling circle amplification (RCA) product. RCA may comprise contacting the biological sample with one or more probes (e.g., a circular probe or circularizable probe or probe set) to produce an RCA product (RCP). RCA, and RCPs thereof, as described in Section II.A. In some embodiments, the nucleic acid concatemer is generated in situ in the biological sample. In some embodiments, the one or more probes or probe sets comprise a circular probe, or a circularizable probe or probe set. In some embodiments, the circular probe or the circularizable probe or probe set is or comprises an oligonucleotide (e.g., as described in Section II.A.(i).).

The nucleic acid concatemer may be generated prior to, during, and/or after contacting the biological sample with a polymerization agent. In some embodiments, the nucleic acid concatemer is generated prior to contacting the biological sample with the polymerization agent. In some embodiments, the nucleic acid concatemer is generated during the contacting the biological sample with the polymerization agent. In some embodiments, the nucleic acid concatemer is generated after contacting the biological sample with the polymerization agent. In some embodiments, the nucleic acid concatemer (e.g., RCP) is generated via RCA prior to, during, and/or after contacting the biological sample with a polymerization agent. In some embodiments, the nucleic acid concatemer is generated via RCA prior to contacting the biological sample with the polymerization agent. In some embodiments, the nucleic acid concatemer is generated via RCA during contacting the biological sample with the polymerization agent. In some embodiments, the nucleic acid concatemer is generated via RCA after contacting the biological sample with the polymerization agent.

The nucleic acid concatemer may be generated prior to, during, and/or after formation of the crosslinked matrix in the biological sample. In some embodiments, the nucleic acid concatemer is generated prior to formation of the crosslinked matrix. In some embodiments, the nucleic acid concatemer is generated during formation of the crosslinked matrix. In some embodiments, the nucleic acid concatemer is generated after formation of the crosslinked matrix. In some embodiments, the nucleic acid concatemer (e.g., RCP) is generated via RCA prior to, during, and/or after formation of the crosslinked matrix in the biological sample. In some embodiments, the nucleic acid concatemer is generated via RCA prior to formation of the crosslinked matrix. In some embodiments, the nucleic acid concatemer is generated via RCA during formation of the crosslinked matrix. In some embodiments, the nucleic acid concatemer is generated via RCA after formation of the crosslinked matrix.

B. Matrix

The present disclosure provides a matrix, e.g., a crosslinked matrix. In some embodiments, the crosslinked matrix does not comprise a plurality of nucleic acid residues (e.g., nucleic acid residues incorporated into the nucleic acid concatemer by a polymerase, such as during amplification) covalently or non-covalently attached thereto.

(i) Polymerization Agent

A crosslinked matrix may be formed by contacting the biological sample with a polymerization agent. For example, the polymerization agent may react with a molecule or product thereof in the biological sample, such as an endogenous cell protein matrix, an exogenous crosslinker, or an exogenous matrix-forming agent, to form the crosslinked matrix. Specifically, the polymerization agent comprises a functional group A capable of reacting with a functional group B in the molecule or product thereof in the biological sample to form the crosslinked matrix.

In some embodiments, the polymerization agent comprises polymerizable monomers or polymers, or cross-linkable polymers. The polymerization agent can form a matrix by polymerization and/or crosslinking of the matrix-forming materials and/or crosslinking agents using methods specific for the matrix forming materials and methods, reagents and conditions. The polymerization agent may form a crosslinked matrix. In some embodiments, the matrix-forming material forms a polyelectrolyte gel. In some embodiments, the polymerization agent forms a hydrogel gel matrix.

In some embodiments, the polymerization agent is a matrix-forming agent. The matrix-forming agent does not necessarily crosslink molecules in the biological sample (e.g., a hydrogel). In some embodiments, the crosslinked matrix comprises moieties of the matrix-forming agent covalently bound to themselves. In some embodiments, the polymerization agent is a crosslinking agent. The crosslinking agent may crosslink molecules in the biological sample. In some embodiments, the crosslinking agent comprises a bivalent or trivalent: thiol, epoxide, amine, alcohol, hydrazide, hydroxylamine, or isocyanate. In some embodiments, the crosslinking agent comprises moieties of the crosslinking agent covalently bound to themselves. In some embodiments, the crosslinking agent comprises moieties of the crosslinking agent covalently bound to endogenous molecules or products thereof in the biological sample.

In some embodiments, the biological sample is in a hydrogel via cross-linking of the polymerization agent that forms the hydrogel. In some embodiments, cross-linking can be performed chemically and/or photochemically. In some embodiments, hydrogel formation occurs within a biological sample. In some embodiments, a biological sample (e.g., tissue section) is embedded in a polymerization agent (e.g., a hydrogel). In some embodiments, hydrogel subunits are infused into the biological sample, and polymerization of the hydrogel is initiated by an external or internal stimulus. In some aspects, a hydrogel may include a macromolecular polymer gel including a network. Within the network, some polymer chains can optionally be cross-linked, although cross-linking does not always occur.

In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

The polymerization agent comprises a functional group A. In some embodiments, the polymerization agent is bifunctional. In some embodiments, the the polymerization agent comprises two or more functional groups A per molecule. In some embodiments, the polymerization agent comprises one or more spacers between functional groups A. In some embodiments, the spacer comprises at least one group selected from C₁₋₆ alkyl, C₂₋₆ alkenyl, C₂₋₆ alkynyl, C═O, O, S, NH, —(C═O)O—, —(C═O)NH—, —S—S—, ethylene glycol, polyethyleneglycol (PEG), propylene glycol, and polypropyleneglycol, or any combination thereof. In some embodiments, the polymerization agent is a crosslinking agent. In some embodiments, the polymerization agent is a crosslinking agent that comprises one or more N-succinimidyloxy groups. In some embodiments, the polymerization agent is a bis(sulfosuccinimidyl) crosslinking agent. In some embodiments, the polymerization agent is bis(sulfosuccinimidyl)suberate (BS3). In some embodiments, the polymerization agent is PEGylated bis(sulfosuccinimidyl)suberate, i.e., BS(PEG)₉.

In some embodiments, the polymerization agent forms a crosslinked matrix (e.g., the polymerization agent is a matrix-forming agent). In some embodiments, the polymerization agent (e.g., the matrix-forming agent) forms a crosslinked matrix that comprises moieties of the polymerized agent bound to themselves. In some embodiments, the polymerization agent (e.g., the matrix-forming agent) forms a crosslinked matrix that comprises moieties of the polymerized agent covalently bound to themselves. In some embodiments, the polymerization agent (e.g., the matrix-forming agent) forms a crosslinked matrix that comprises moieties of the polymerized agent non-covalently bound to themselves. In some embodiments, the polymerization agent comprises poly(acrylate), poly(alkyl-acrylate), poly(hydroxyalkyl-acrylate), poly(acrylamide), poly(alkyl-acrylamide), poly(ethylene glycol), poly(vinyl alcohol), poly(vinyl pyrrolidone), poly(lactic acid), poly(lactic-co-glycolic acid), hyaluronic acid, chitosan, heparin, alginate, fibrin, collagen, gelatin, copolymers of any two or more of the foregoing, or combinations thereof.

In some embodiments, the functional group A of the polymerization agent is capable of chemically attaching to a specific functional group B in a molecule or product thereof in the biological sample, to form the crosslinked matrix. In some embodiments, the functional group B is in molecules of an endogenous cell protein matrix in the biological sample. In some embodiments, the functional group B is in molecules of an exogenous crosslinker or matrix-forming agent, such as any of the crosslinking agents or matrix-forming agents described herein. In some embodiments, the crosslinked matrix is formed from a click reaction between functional group A of the polymerization agent and functional group B in the molecule or product thereof in the biological sample. In some embodiments, the click reaction is biorthogonal. In some embodiments, the click reaction is orthogonal to one or more enzymatic reactions in the biological sample. In some embodiments, the functional groups that participate in the click reaction (e.g., functional group A and functional group B) may comprise, but are not limited to, azido/alkynyl; azido/cyclooctynyl; tetrazine/dienophile; thiol/alkynyl; cyano/1,2-amino thiol; and, nitrone/cyclooctynyl.

In some embodiments, the reaction between functional group A of the polymerization agent and functional group B in the molecule or product thereof in the biological sample is photo-initiated. In some embodiments, the reaction between functional group A of the polymerization agent and functional group B in the molecule or product thereof in the biological sample is thermally initiated. In some embodiments, the reaction between functional group A of the polymerization agent and functional group B in the molecule or product thereof in the biological sample is a photo-initiated click reaction. In some embodiments, the reaction between functional group A of the polymerization agent and functional group B in the molecule or product thereof in the biological sample is a thermally initiated click reaction. In some embodiments, the reaction between functional group A of the polymerization agent and functional group B in the molecule or product thereof in the biological sample is a catalyzed reaction. In some embodiments, the reaction between functional group A of the polymerization agent and functional group B in the molecule or product thereof in the biological sample is a copper-catalyzed reaction. In some embodiments, the reaction between functional group A of the polymerization agent and functional group B in the molecule or product thereof in the biological sample is a copper-catalyzed click reaction. In some embodiments, the reaction between functional group A of the polymerization agent and functional group B in the molecule or product thereof in the biological sample occurs under ambient conditions.

(ii) Crosslinked Matrix

The polymerization agent may form a crosslinked matrix in a biological sample, comprising a nucleic acid concatemer (e.g., a RCP) or a molecule for generating the nucleic acid concatemer, while confining the nucleic acid concatemer or the molecule for generating the nucleic acid concatemer (and the resultant nucleic acid concatemer) in the biological sample and/or the crosslinked matrix. In some embodiments, the nucleic acid concatemer is confined within the biological sample. In some embodiments, the nucleic acid concatemer is confined within the crosslinked matrix. In some embodiments, the nucleic acid concatemer is confined within a space within the crosslinked matrix and/or the biological sample. The nucleic acid concatemer may be confined within the crosslinked matrix by co-polymerization of the molecule for generating the nucleic acid concatemer in the biological sample with the polymerization agent. The nucleic acid concatemer may also be confined within the crosslinked matrix by crosslinking of the molecule for generating the nucleic acid concatemer in the biological sample with the polymerization agent. The nucleic acid concatemer may also be confined within the crosslinked matrix by covalent attachment of the molecule for generating the nucleic acid concatemer in the biological sample with the polymerization agent. In some embodiments, the crosslinked matrix is covalently bound to the nucleic acid concatemer at a primer sequence that is used to prime RCA to generate the nucleic acid concatemer (e.g., RCP).

In some embodiments, the crosslinked matrix is porous, thereby permitting the introduction of reagents into the crosslinked matrix at the site of a molecule for generating a nucleic acid concatemer (e.g., for amplification of a nucleic acid). A porous crosslinked matrix may be made. For example, a polyacrylamide gel matrix may be co-polymerized with acrydite-modified streptavidin monomers and biotinylated DNA molecules, using a suitable acrylamide:bis-acrylamide ratio to control the cross-linking density. In some embodiments, control over the molecular sieve size and density is achieved by adding additional cross-linkers such as functionalized polyethylene glycols.

In some embodiments, the crosslinked matrix is sufficiently optically transparent and/or may have optical properties compatible with the detection and analysis techniques described herein (see, e.g., Section IV), such as standard sequencing chemistries and/or imaging.

In some aspects, the crosslinked matrix is charge neutral. In some embodiments, the crosslinked matrix does not have a positive or negative charge. Charges may be introduced into the crosslinked matrix, for example, by the polymerization agent (e.g., crosslinking agent or matrix-forming agent) and/or other modifications during formation of the crosslinked matrix. In some embodiments, the crosslinked matrix is positively charged. In some examples, the matrix is generated with negatively charged agents incorporated. In a positively charged crosslinked matrix, nucleic acid concatemers may be confined by interacting with the positive charges on the crosslinked matrix. In some embodiments, the crosslinked matrix is negatively charged. In a negatively charged crosslinked matrix, nucleic acid concatemers may be confined by repulsion from the crosslinked matrix, thereby resulting in the nucleic acid concatemers stacking on top of one another.

In some embodiments, a nucleic acid concatemer is confined in the biological sample. In some embodiments, the nucleic acid concatemer is confined in the crosslinked matrix. In some embodiments, the diameter of the confined nucleic acid concatemer is smaller than that of a reference nucleic acid concatemer that is generated in a biological sample not comprising a crosslinked matrix. In some embodiments, the diameter of a RCA product confined in the crosslinked matrix is between about 0.3 μm and about 1 μm less than that of the nucleic acid concatemer in the reference biological sample, such as between any of about 0.3 μm and about 0.7 μm, about 0.5 μm and about 0.9 μm, and about 0.6 μm and about 1 μm, and is less than that of the nucleic acid concatemer in the reference biological sample.

In some embodiments, a plurality of nucleic acid concatemers are confined in the biological sample. In some embodiments, the average diameter of the confined nucleic acid concatemers is smaller than that of reference nucleic acid concatemers that are generated in a biological sample not comprising a crosslinked matrix. For example, in some embodiments, the average diameter of the confined nucleic acid concatemers is between about 5% smaller and about 25% smaller than that of the nucleic acid concatemers in the reference biological sample, such as between any of about 5% smaller and about 15% smaller, about 10% smaller and about 20% smaller, and about 15% smaller and about 25% smaller than that of the nucleic acid concatemers in the reference biological sample. In some embodiments, the average diameter of the confined nucleic acid concatemers is at least about 5% smaller, at least about 10% smaller, at least about 15% smaller, at least about 20% smaller, at least about 25% smaller, or more than about 25% smaller than that of the nucleic acid concatemers in the reference biological sample. In some embodiments, the average diameter of the confined nucleic acid concatemers is less than about 25% smaller, less than about 20% smaller, less than about 15% smaller, less than about 10% smaller, or less than about 5% smaller than that of the nucleic acid concatemers in the reference biological sample.

In some aspects, the average distance between the confined nucleic acid concatemers is larger than that of reference nucleic acid concatemers that are generated in a reference biological sample not comprising a crosslinked matric. For example, in some embodiments, the average distance between the confined nucleic acid concatemers is at least between about 1 nucleic acid concatemer diameter and about 2 nucleic acid concatemers diameter, between about 2 nucleic acid concatemer diameter and about 5 nucleic acid concatemers diameter, or between about 3 nucleic acid concatemer diameter and about 10 nucleic acid concatemers diameter. In some embodiments, the average distance between the confined nucleic acid concatemers is between about 5% greater and about 25% greater than that of the reference nucleic acid concatemers generated in a reference biological sample not comprising a crosslinked matric, such as between any of about 5% greater and about 15% greater, about 10% greater and about 20% greater, and about 15% greater and about 25% greater than that of the nucleic acid concatemers in the reference biological sample. In some embodiments, the average distance between the confined nucleic acid concatemers is at least about 5% greater, at least about 10% greater, at least about 15% greater, at least about 20% greater, at least about 25% greater, or more than about 25% greater than that of the nucleic acid concatemers in the reference biological sample. In some embodiments, the average distance between the confined nucleic acid concatemers is less than about 25% greater, less than about 20% greater, less than about 15% greater, less than about 10% greater, or less than about 5% greater than that of the nucleic acid concatemers in the reference biological sample

In some aspects, the average diameter of the confined nucleic acid concatemers is between about 0.3 μm and about 1 μm, such as between any of about 0.3 μm and about 0.7 μm, about 0.5 μm and about 0.9 μm, and about 0.6 μm and about 1 μm. In some embodiments, the average diameter of the confined nucleic acid concatemers is less than about 1 μm, less than about 0.9 μm, less than about 0.8 μm, less than about 0.7 μm, less than about 0.6 μm, less than about 0.5 μm, less than about 0.4 μm, less than about 0.3 μm, or smaller. In some embodiments, the average diameter of the confined nucleic acid concatemers is greater than about 0.3 μm, greater than about 0.4 μm, greater than about 0.5 μm, greater than about 0.6 μm, greater than about 0.7 μm, greater than about 0.8 μm, greater than about 0.9 μm, greater than about 0.1 μm, or bigger. In some aspects, the average diameter of the confined nucleic acid concatemers in the crosslinked sample (e.g., crosslinked after probe hybridization to generate a crosslinked matrix in the sample prior to RCA) is between about 0.2 μm and about 0.8 μm, whereas the average diameter of the reference nucleic acid concatemers generated in a reference sample not comprising the crosslinked matrix is more than 0.8 μm, more than 0.9 μm, more than 1 μm, more than 1.1 μm, more than 1.2 μm, more than 1.3 μm, more than 1.4 μm, or more than 1.5 μm.

In some aspects, the average length and/or volume of the confined nucleic acid concatemers is reduced compared to that of a reference nucleic acid concatemer that is not in a biological sample comprising a crosslinked matrix. In some embodiments, the average length of the confined nucleic acid concatemers is between about 1 kb and about 50 kb, such as between any of about 1 kb and about 10 kb, about 5 kb and about 15 kb, and about 10 kb and about 25 kb. In some embodiments, the average length of the confined nucleic acid concatemers is more than about 1 kb, more than about 5 kb, more than about 10 kb, more than about 25 kb, more than about 50 kb, or longer. In some embodiments, the average length of the confined nucleic acid concatemers is less than about 50 kb, less than about 25 kb, less than about 10 kb, less than about 5 kb, less than about 1 kb, or shorter.

### III. Samples, Analytes, and Target Sequences

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable (e.g., padlock) probe.

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

(viii) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labeling Agents

In some embodiments, provided herein are methods and compositions for analyzing endogenous analytes (e.g., RNA, ssDNA, and cell surface or intracellular proteins and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

C. Target Sequences

A target sequence for a probe disclosed herein may be comprised in any analyte disclose herein, including an endogenous analyte (e.g., a viral or cellular nucleic acid), a labeling agent, or a product of an endogenous analyte and/or a labeling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labeled probes (e.g., detection oligos). In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., US 2019/0055594 and US 2021/0164039, which are hereby incorporated by reference in their entireties.

### IV. Detection and Analysis

In some aspects, the provided methods involve analyzing, e.g., detecting or determining, one or more sequences present in a molecule for generating a nucleic acid concatemer and/or in the nucleic acid concatemers described herein. In some embodiments, a target nucleic acid is present in the molecule generating a nucleic acid concatemer and/or in the nucleic acid concatemers described herein. In some embodiments, the detecting comprises hybridizing one or more detectably labeled probes to the nucleic acid concatemer, or via hybridization to adaptor probes that hybridize to the nucleic acid concatemer). In some embodiments, the analysis comprises determining the sequence of all or a portion of the nucleic acid concatemer (e.g., a barcode sequence or a complement thereof), wherein the sequence is indicative of a sequence of the target nucleic acid.

In some cases, analysis is performed on one or more images captured, and may comprise processing the image(s) and/or quantifying signals observed. In some embodiments, images of signals from different fluorescent channels and/or detectable probe hybridization cycles can be compared and analyzed. In some embodiments, images of signals (or absence thereof) at a particular location in a sample from different fluorescent channels and/or sequential detectable probe hybridization cycles can be aligned to analyze an analyte at the location. For instance, a particular location in a sample can be tracked and signal spots from sequential probe hybridization (and optionally ligation) cycles can be analyzed to detect a target polynucleotide sequence (e.g., a barcode sequence or subsequence thereof) in a nucleic acid at the location. The analysis may comprise processing information of one or more cell types, one or more types of analytes, a number or level of analyte, and/or a number or level of cells detected in a particular region of the sample. In some embodiments, the analysis comprises detecting a sequence e.g., a barcode sequence present in an amplification product at a location in the sample. In some embodiments, the analysis includes quantification of puncta (e.g., if amplification products are detected). In some cases, the analysis includes determining whether particular cells and/or signals are present that correlate with one or more analytes from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization in a FISH-type assay, sequencing by hybridization).

In some embodiments, the methods comprise sequencing all or a portion of the nucleic acid concatemer, such as one or more barcode sequences present in the nucleic acid concatemer. In some embodiments, the sequence of the nucleic acid concatemer, or barcode thereof, is indicative of a sequence of the target nucleic acid to which the nucleic acid concatemer is hybridized. In some embodiments, the analysis and/or sequence determination comprises sequencing all or a portion of the nucleic acid concatemer and/or in situ hybridization to the nucleic acid concatemer. In some embodiments, the sequencing step involves sequencing by hybridization, sequencing by ligation, sequencing by synthesis, sequencing by binding, and/or fluorescent in situ sequencing (FISSEQ), hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US2017/0009278, which is incorporated herein by reference, for exemplary probes and HCR reaction components. In some embodiments, the detection or determination comprises hybridizing to the first overhang a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is an amplification product (e.g., a rolling circle amplification product/nucleic acid concatemer).

In some aspects, the provided methods comprise imaging the probe hybridized to the nucleic acid concatemer, for example, via binding of a detectably labeled probe and detecting the detectable label. In some embodiments, the detection probe comprises a detectable label that can be measured and quantitated. The terms “label” and “detectable label” comprise a directly or indirectly detectable moiety that is associated with (e.g., conjugated to) a molecule to be detected, e.g., a probe that is a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some embodiments, sequence analysis of nucleic acids (e.g., nucleic acids such as probes or RCA products comprising barcode sequences generated using the circular nucleic acid as template) can be performed by sequential hybridization (e.g., sequencing by hybridization and/or sequential in situ fluorescence hybridization). Sequential fluorescence hybridization can involve sequential hybridization of detectable probes comprising an oligonucleotide and a detectable label. In some embodiments, a method disclosed herein comprises sequential hybridization of the detectable probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectable probes are described in US 2019/0161796, US 2020/0224244, US 2022/0010358, US 2021/0340618, and US 2023/0039899, all of which are incorporated herein by reference in their entireties. In some embodiments, the methods provided herein can include analyzing the identifier sequences (e.g., analyte sequences or barcode sequences) by sequential hybridization and detection with a plurality of labeled probes (e.g., detection oligonucleotides).

In some embodiments, sequence detection comprises contacting the biological sample with one or more intermediate probes that directly or indirectly hybridize to the rolling circle amplification product, wherein the one or more intermediate probes are detectable using one or more detectably-labeled probes, and dehybridizing the one or more intermediate probes and/or the one or more detectably-labeled probes from the rolling circle amplification product. In some embodiments, the one or more intermediate probes comprise one or more overhang regions (e.g., a 5′ and/or 3′ end of the probe that does not hybridize to the rolling circle amplification product). A probe comprising a single overhang region may be referred to as an “L-shaped probe,” and a probe comprising two overhangs may be referred to as a “U-shaped probe.” In some cases, the overhang region comprises a binding region for binding one or more detectably-labeled probes. In some embodiments, the detecting comprises contacting the biological sample with a pool of intermediate probes corresponding to different barcode sequences or portions thereof, and a pool of detectably-labeled probes corresponding to different detectable labels. In some embodiments, the biological sample is sequentially contacted with different pools of intermediate probes. In some instances, a common or universal pool of detectably-labeled probes is used in a plurality of sequential hybridization steps (e.g., with different pools of intermediate probes).

In some embodiments, provided herein are methods for in situ analysis of analytes in a sample using sequential probe hybridization. In some aspects provided herein is a method for analyzing a biological sample, comprising: a) generating a rolling circle amplification product (RCP) of a circular probe or circularizable probe or probe set described herein, the RCP comprising an identifier sequence such as a barcode sequence or analyte sequence, wherein the identifier sequence is associated with an analyte of interest and is assigned a signal code sequence; b) contacting the biological sample with a first probe (e.g., an intermediate probe such as an L-probe) and a first detectably labeled probe to generate a first complex comprising the first probe hybridized to the RCP and the first detectably labeled probe hybridized to the first probe, wherein the first probe comprises (i) a recognition sequence (e.g., a target-binding sequence) complementary to the identifier sequence (e.g., barcode sequence or analyte sequence) and (ii) a first landing sequence (e.g., an overhang sequence), and wherein the first detectably labeled probe comprises a sequence complementary to the first landing sequence; c) detecting a first signal associated with the first detectably labeled probe, wherein the first signal corresponds to a first signal code in the signal code sequence; d) contacting the biological sample with a second probe (e.g., an intermediate probe such as L-probe) and a second detectably labeled probe to generate a second complex comprising the second probe hybridized to the RCP and the second detectably labeled probe hybridized to the second probe, wherein the second probe comprises (i) a recognition sequence (e.g., a target-binding sequence) complementary to the identifier sequence (e.g., barcode sequence or analyte sequence) and (ii) a second landing sequence (e.g., an overhang sequence), and wherein the second detectably labeled probe comprises a sequence complementary to the second landing sequence; and e) detecting a second signal associated with the second detectably labeled probe, wherein the second signal corresponds to a second signal code in the signal code sequence, wherein the signal code sequence comprising the first signal code and the second signal code is determined at a location in the biological sample, thereby decoding the identifier sequence (e.g., barcode sequence or analyte sequence) and identifying the analyte of interest at the location in the biological sample. In some embodiments, the detectable label of the first detectably labeled probe and the detectable label of the second detectably labeled probe are the same. In some embodiments, the detectable labels of the first detectably labeled probe and the second detectably labeled probe are different. In some embodiments, the first signal code and the second signal code are the same. In some embodiments, the first signal code and the second signal code are different.

In some embodiments, the first probe (e.g., a first intermediate probe such as a first L-probe), the second probe (e.g., a second intermediate probe such as a second L-probe), and one or more subsequent probes (e.g., subsequent intermediate probe such as subsequent L-probes) are contacted with the biological sample sequentially in a pre-determined sequence which corresponds to the signal code sequence assigned to the identifier sequence (e.g., barcode sequence or analyte sequence), wherein the one or more subsequent probes each comprises (i) a recognition sequence complementary to the identifier sequence (e.g., barcode sequence or analyte sequence) and (ii) an overhang sequence complementary to a detectably labeled probe of a pool (e.g., a universal pool across different cycles of probe hybridization) of detectably labeled probes. In some embodiments, the biological sample is contacted with the first probe before the second probe and one or more subsequent probes. In some embodiments, the biological sample is contacted with the second after the first probe and before and one or more subsequent probes. In some embodiments, the biological sample is contacted with the one or more subsequent probes after the first probe. In some embodiments, the biological sample is contacted with the one or more subsequent probes after the first probe and the second probe.

In some embodiments, the first detectably labeled probe and the second detectably labeled probe are in the pool of detectably labeled probes. A pool of detectably labeled probes may comprises at least two detectably labeled probes, and may be used for multiplexing analyses of two or more target analytes (e.g., target nucleic acids) in a biological sample. In some embodiments, the contacting in b) comprises contacting the biological sample with the universal pool of detectably labeled probes, and the contacting in d) comprises contacting the biological sample with the universal pool of detectably labeled probes. In some embodiments, the universal pool of detectably labeled probes used in the contacting in b) is the same as the universal pool of detectably labeled probes used in the contacting in d). In some embodiments, the universal pool comprises detectably labeled probes each having a detectable label corresponding to a different nucleic acid sequence for hybridization to a landing sequence (e.g., an overhang sequence) in a probe (e.g., an intermediate probe such as an L-probe). In some embodiments, the number of different detectably labeled probes in the universal pool is four.

In some embodiments, the one or more subsequent probes are contacted with the biological sample to determine signal codes in the signal code sequence until sufficient signal codes have been determined to decode the identifier sequence (e.g., barcode sequence or analyte sequence), thereby identifying the target analyte (e.g., target nucleic acid). In some embodiments, the method further comprises a step of removing the first probe and/or the first detectably labeled probe from the biological sample before contacting the sample with a subsequent probe and a detectably labeled probe hybridizing to the subsequent probe. In some embodiments, the method further comprises a step of removing the second probe and/or the second detectably labeled probe from the biological sample, before contacting the sample with a subsequent probe and a detectably labeled probe hybridizing to the subsequent probe.

In some embodiments, the method further comprises identifying multiple different target analytes present at locations (e.g., different locations) in the biological sample. In some embodiments, each different target analyte is assigned a different signal code sequence and is targeted by a circular or circularizable probe or probe set comprising a complement of a different barcode sequence of the plurality of barcode sequences. In some embodiments, the number of different probes (e.g., L-probes that have different recognition sequences that bind to the barcode sequences) in each pool of probes is greater than the number of different detectably labeled probes in the universal pool of detectably labeled probes. In some embodiments, the number of different detectably labeled probes in the universal pool is four. In some embodiments, the number of different probes in each pool of probes (e.g., L-probes) is about 10, about 20, about 30, about 40, about 50, about 100, about 200, about 500, about 1,000, or more. In some embodiments, the number of different recognition sequences (e.g., recognition sequences that bind to the barcode sequences) of probes in each pool of probes in at least about 10, such as at least any of about 20, 30, 40, 50, 100, 200, 500, 1,000, or more.

In some embodiments, a method disclosed herein may also comprise one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, the RCA product can be detected in with a method that comprises signal amplification.

In some embodiments, the RCA product (e.g., generated as described in Section II) can be detected by providing detection probes. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the polynucleotide probes.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more nucleic acid concatemer(s) described herein. In some embodiments, the methods involve incubating the detectable probe containing the detectable label with the sample, washing unbound detectable probe, and detecting the label, e.g., by imaging. In some embodiments, the nucleic acid concatemer(s) remain crosslinked to the target nucleic acid during the washing and detecting steps.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264.

As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores include those described in Henegariu et al. (2000) Nature Biotechnol. 18:345.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as an Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequence determination can be performed in situ. In situ sequence determination typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932. Exemplary techniques for in situ sequencing comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121).

In some embodiments, sequence determination can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232.

In some embodiments, sequence determination can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597.

In some embodiments, the barcodes of the detection probes are targeted by detectably labeled oligonucleotides, such as fluorescently labeled oligonucleotides. In some embodiments, one or more decoding schemes are used to decode the signals, such as fluorescence, for sequence determination. In any of the embodiments herein, barcodes can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labeled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; US2020/0080139 A1; US20210017587 A1; and US 2017/0220733 A1, all of which are incorporated by reference in their entireties. In some embodiments, these assays enable signal amplification, combinatorial decoding, and error correction schemes at the same time.

In some embodiments, nucleic acid hybridization can be used for sequence determination. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### V. Compositions, Kits, and Systems

In some embodiments, disclosed herein is a composition that comprises a complex containing a target nucleic acid (e.g., a target nucleic amplification product, such as a nucleic acid concatemer, e.g., a rolling circle amplification product) and a crosslinked matrix in a biological sample, e.g., any of the target nucleic acids and crosslinked matrixes described in Section II. In some embodiments, the complex further comprises a detectable probe, e.g., as described in Section IV. The polymerized network confines the nucleic acid concatemer in the biological sample and/or the crosslinked matrix.

Also provided herein are kits, for example comprising one or more polymerization agents, e.g., any described in Section II, and instructions for performing the methods provided herein. In some embodiments, the kit comprises a substrate for forming a crosslinked matrix. and one or more second materials. In some embodiments, kit comprises one or more structures (e.g., wells, ridges, projections, and/or markings) located on a substrate for generated the crosslinked matrix comprising the biological sample.

In some embodiments, the kits further comprise one or more reagents required for one or more steps comprising hybridization, ligation, extension, detection, sequencing, and/or sample preparation as described herein. In some embodiments, the kit further comprises a target nucleic acid, e.g., any described in Sections II and III. In some embodiments, any or all of the oligonucleotides are DNA molecules. In some embodiments, the target nucleic acid is a messenger RNA molecule. In some embodiments, the target nucleic acid is a probe (e.g., a padlock probe, such as a molecule for generating a nucleic acid concatemer) or an amplification product thereof (e.g., a rolling circle amplification product, such as a nucleic acid concatemer). The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers. In some aspects, the kit can also comprise any of the reagents described herein, e.g., wash buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, and reagents for additional assays.

### VI. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to confine a nucleic acid concatemer in a biological sample via a polymerization agent that may form a crosslinked matrix in the biological sample, to increase the resolution and stability of the nucleic acid concatemers in situ.

In some embodiments, the region of interest comprises a single-nucleotide polymorphism (SNP). In some embodiments, the region of interest comprises is a single-nucleotide variant (SNV). In some embodiments, the region of interest comprises a single-nucleotide substitution. In some embodiments, the region of interest comprises a point mutation. In some embodiments, the region of interest comprises a single-nucleotide insertion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. An estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif; HeliScope™ by Helicos Biosciences Corporation, Cambridge, Ma.; and PacBio RS by Pacific Biosciences of California, Inc., Menlo Park, Calif), sequencing by ion detection technologies (such as Ion Torrent™ technology, Life Technologies, Carlsbad, Calif); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“Multiplexing” or “multiplex assay” herein may refer to an assay or other analytical method in which the presence and/or amount of multiple targets, e.g., multiple nucleic acid target sequences, can be assayed simultaneously by using more than one probe, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

As used herein, the term “alkyl”, by itself or as part of another substituent, means, unless otherwise stated, a straight or branched chain hydrocarbon radical, having the number of carbon atoms designated (e.g., C₁-C₆ means one to six carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the term “alkyl” may encompass C₁-C₆ alkyl, C₂-C₆ alkyl, C₃-C₆ alkyl, C₄-C₆ alkyl, C₅-C₆ alkyl, C₁-C₅ alkyl, C₂-C₅ alkyl, C₃-C₅ alkyl, C₄-C₅ alkyl, C₁-C₄ alkyl, C₂-C₄ alkyl, C₃-C₄ alkyl, C₁-C₃ alkyl, C₂-C₃ alkyl, or C₁-C₂ alkyl.

As used herein, the term “alkenyl”, by itself or as part of another substituent, means, unless otherwise stated, a straight or branched chain hydrocarbon radical having one or more double bond functional groups (e.g., a —CH═CH— moiety), having the number of carbon atoms designated (e.g., C₂-C₆ means two to six carbons). An alkenyl group may comprise 1, 2, 3, 4, 5 or 6 or more double bond functional groups (e.g., a —CH═CH— moiety). An alkenyl group having multiple double bonds may have the double bonds in conjugation (e.g., a 1,3-butadienyl group) or not in conjugation (e.g., with one or more intervening saturated carbon atoms). Examples of alkenyl groups include ethenyl (e.g., vinyl), propenyl, allyl, isopropenyl, butenyl, pentenyl, hexenyl, and the like.

As used herein, the term “alkynyl”, by itself or as part of another substituent, means, unless otherwise stated, a straight or branched chain hydrocarbon radical having one or more triple bond functional groups (e.g., a —C≡C— moiety), having the number of carbon atoms designated (e.g., C₂-C₆ means two to six carbons). An alkynyl group may comprise 1, 2, 3, 4, 5 or 6 or more double bond functional groups (e.g., a —C≡C— moiety). An alkynyl group having multiple triple bonds may have the triple bonds in conjugation (e.g., a polyacetylene) or not in conjugation (e.g., with one or more intervening saturated carbon atoms). Examples of alkenyl groups include ethynyl, propynyl, butynyl, pentynyl, hexynyl, and the like.

As used herein, “poly(ethylene glycol)” (“PEG”, “polyethylene glycol”, or “polyethyleneglycol”) refers to a polymer or polymer moiety having a repetition of ethylene glycol units of the formula

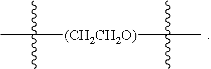

PEGs include polydisperse PEGs and monodisperse PEGs. Polydisperse PEGs are characterized by a distribution of sizes and molecular weights, whereas monodisperse PEGs are typically purified polymers or polymer moieties of a single chain length and molecular weight.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Confining a Nucleic Acid Concatemer in a Biological Sample Using a Polymerization Agent

This example demonstrates a method for confining a nucleic acid concatemer in a biological sample using a polymerization agent, e.g., a crosslinking agent such as BS3 (bis(sulfosuccinimidyl)suberate) or BS(PEG)₉ (PEGylated bis(sulfosuccinimidyl)suberate). In particular, this example demonstrates confining a nucleic acid concatemer via a crosslinked matrix formed using a polymerization agent, wherein the crosslinked matrix is not covalently bound to nucleic acid residues incorporated into the nucleic acid concatemer by a polymerase.

A biological sample (e.g., a processed or cleared biological sample, a tissue sample, a sample embedded in a hydrogel, etc.) is contacted with one or more polymerization agents. For example, the one or more polymerization agents may be one or more matrix-forming agents (e.g., one or more three-dimensional matrix forming agents) or one or more crosslinking agents. The matrix may comprise moieties of a matrix-forming agent covalently bound to themselves, moieties of the matrix-forming agent covalently bound to one or more other matrix-forming agents, and/or moieties of the matrix-forming agent covalently bound to endogenous molecules or products thereof in the biological sample. In some examples, the matrix may comprise moieties of a crosslinking agent covalently bound to themselves, moieties of the crosslinking agent covalently bound to one or more other matrix-forming agents, and/or moieties of the crosslinking agent covalently bound to endogenous molecules or products thereof in the biological sample.

A molecule for generating a nucleic acid concatemer may be present in the biological sample prior to contacting the sample with the polymerization agent (e.g., a crosslinker such as BS3 or BS(PEG)₉). For instance, the molecule for generating the nucleic acid concatemer can be an endogenous molecule in the sample, such as genomic DNA or a cellular RNA, or a product of the endogenous molecule. The product can be for example cDNA. In other examples, the molecule (e.g., for use as a template) for generating the nucleic acid concatemer can be an exogenous molecule, such as a synthetic nucleic acid probe introduced into the sample prior to sample crosslinking.

Alternatively, a molecule for generating a nucleic acid concatemer may be contacted with the biological sample during or after contacting the sample with the polymerization agent, or during or after sample crosslinking using the polymerization agent.

Regardless of whether the molecules for generating a nucleic acid concatemer (e.g., RCA template and/or primer) are present before or after sample crosslinking using the polymerization agent, the nucleic acid concatemer in this example is not generated until after the sample crosslinking. In some examples, the nucleic acid concatemer is generated in the crosslinked sample while the space that it occupies at the end of the reaction (e.g., RCA) is limited by the crosslinked matrix preformed prior to the amplification reaction.

The polymerization agent can comprise a functional group A that is capable of reacting with a functional group B in a molecule or product thereof in the biological sample to form a crosslinked matrix. Furthermore, the crosslinked matrix is not covalently bound to nucleic acid residues incorporated into a nucleic acid concatemer. In some examples, a polymerization agent that is a crosslinking agent may crosslink an RCA primer comprising functional group B to functional group A of the crosslinking agent. Prior to RCA, the crosslinked matrix is formed by reaction of functional group A of the polymerization agent with functional group B in a molecule or product thereof in the biological sample (such as the RCA primer and/or endogenous molecules in the sample, e.g., proteins, nucleic acids, lipids, carbohydrates, and/or minerals). The crosslinked matrix provides limited space therein for the generation of a nucleic acid concatemer, and the resultant nucleic acid concatemer can be confined in the biological sample and/or the crosslinked matrix.

Overall, limiting the generation of a nucleic acid concatemer within a biological sample using a crosslinked matrix can allow for reduced swelling and/or increased compaction of the resulting nucleic acid concatemer, thereby facilitating increased resolution of nucleic acid concatemers in the sample during detection.

### Example 2: Confining RCA Products in a Fixed Biological Sample Crosslinked Prior to RCA

This example demonstrates reducing detected size of RCA products by confining the RCA products in spaces within a crosslinked matrix in a fixed biological sample which is crosslinked prior to RCA to generate the crosslinked matrix.

Fresh frozen mouse brain tissue sections were fixed with PFA and circularizable probes (e.g., padlock probes) were added to the samples in hybridization buffers (e.g., including SSC and formamide) and incubated with the samples to allow hybridization of the circularizable probes to their target nucleic acids. Then, the probe hybridization mixture was removed and the samples were washed. For ligation of the circularizable probes hybridized to their target nucleic acids, a ligation reaction mix (e.g., containing a SplintR® ligase buffer, RNase inhibitor and SplintR® ligase) was added and the samples were incubated for probe circularization. An RCA primer was added to the samples to hybridize to the probes. The samples were then contacted with bis(sulfosuccinimidyl)suberate (BS3) in a phosphate buffered saline (PBS) buffer or with PBS only (no BS3) as a control. The fixed tissue sections were then subjected to crosslinking conditions, and a crosslinked matrix was formed in tissue samples in the BS3 groups (BS3-1 and BS3-2) but not in the PBS groups (PBS-1 and PBS-2). This step of BS3 fixation/crosslinking was done after probe ligation and RCA primer hybridization. The RCA primers were crosslinked to the crosslinked matrix with BS3 (although crosslinking the RCA primers to the crosslinked matrix was optional) and the BS3 fixation/crosslinking also crosslinked the tissue matrix.

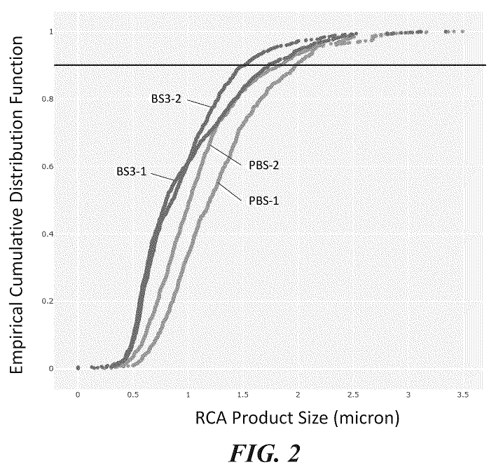

The samples were washed and an RCA reaction mixture (containing Phi29 reaction buffer, dNTPs, Phi29 polymerase) was added and incubated with the samples for overnight to amplify the circularized probes. No modified nucleotides capable of covalent crosslinking by BS3 were incorporated into the RCA products. The sizes of the RCA products are shown in FIG. 2, where samples crosslinked using BS3 prior to RCA showed reduced RCA product sizes compared to the control samples treated with PBS prior to RCA.

These data show that contacting a sample with a crosslinking agent prior to RCA results in reduction in size of the RCA products without covalently binding the RCA products to themselves or to the crosslinked matrix via modified nucleotides incorporated into the RCA products during the RCA reaction.

### Example 3: Confining RCA Products in a Fixed Biological Sample Crosslinked after RCA

Fresh frozen brain tissue samples from a 6 week old mouse were fixed in formaldehyde. The samples were analyzed essentially as described in Example 2, except that the BS3 crosslinking was performed after RCA.

Briefly, the fixed samples were contacted with circularizable probes, which were hybridized at 37° C. overnight. Following hybridization, the samples were washed in SSC/formamide to remove unbound probes from the samples. The probes were ligated into circular probes using a SplintR® ligase polymerase in SplintR® ligase buffer. The samples were contacted with RCA primers and an RCA reaction mixture (containing Phi29 reaction buffer, dNTPs, Phi29 polymerase) and RCA was performed at 37° C. for 2 hours, 1.5 hours, or 1 hour. The samples containing RCA products were then contacted with BS3 in a PBS buffer at room temperature or with PBS only (no BS3) as a control. The samples were washed in PBS, and the crosslinking reaction was inhibited using a Tris buffer at room temperature. BS3 crosslinking created a crosslinked matrix in the BS3-treated samples, whereas control samples treated with PBS only remained un-crosslinked. Since no modified nucleotides capable of covalent crosslinking by BS3 were incorporated during RCA, the RCA products were not crosslinked to themselves and/or to the matrix through the reaction between BS3 and modified nucleotides incorporated during RCA.

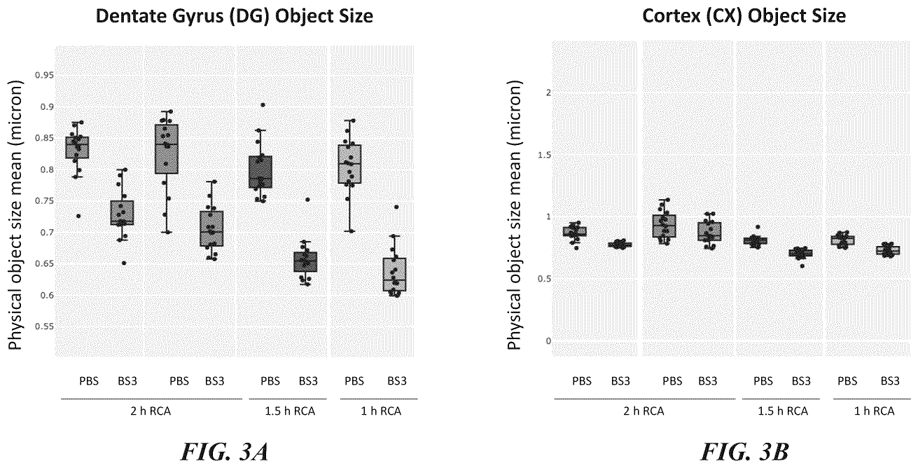

The sizes of the RCA products are shown in FIGS. 3A-3B. RCA products in the samples crosslinked using BS3 after RCA were reduced in size compared to RCA products in the samples treated with PBS after RCA. For all RCA reaction times (2 hours, 1.5 hours, and 1 hour) tested, RCA product sizes were reduced in BS3-crosslinked samples compared to PBS control samples, in the dentate gyrus (DG) (FIG. 3A) and in the cortex (CX) (FIG. 3B). Incorporation of aminoallyl-dUTP during RCA and reacting with BS3 did not further reduce the sizes of the RCA products containing the modified nucleotide residues (data not shown), suggesting it was the crosslinking itself that confined the RCPs and not the crosslinking of the modified nucleotides in the RCPs to itself/to the tissue matrix that was the RCP size reducing factor in this example.

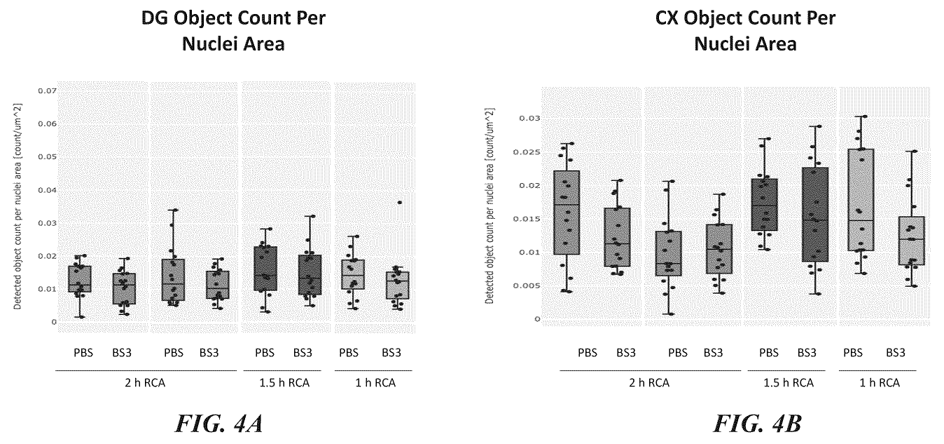

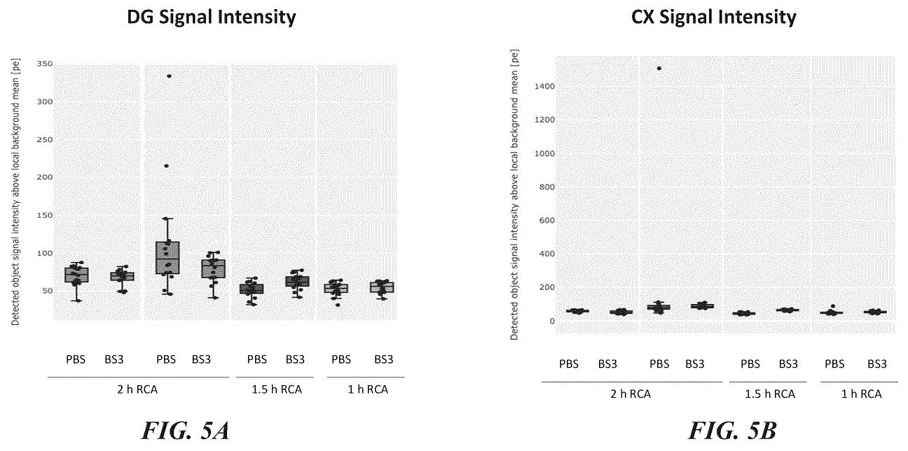

Moreover, despite the reduction in size, the samples crosslinked using BS3 had similar RCA product count per nuclei (FIGS. 4A-4B) and similar signal intensity (FIGS. 5A-5B) compared to the PBS control samples. These data show that contacting a sample with a polymerization agent (such as BS3) to crosslink the sample after RCA results in reduction in size of the RCA products without the need to covalently bind the modified nucleotides incorporated during the RCA to the crosslinked matrix.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for sample analysis, comprising:
(a) contacting a biological sample with a crosslinking agent to form a crosslinked matrix in the biological sample, wherein the biological sample is fixed prior to contacting the crosslinking agent; and
(b) using a polymerase to generate a rolling circle amplification (RCA) product in the crosslinked matrix, wherein the crosslinked matrix is not covalently bound to nucleic acid residues of the RCA product.

2. The method of claim 1, wherein the crosslinking agent is a non-nucleic acid molecule.

3. The method of claim 1 or 2, wherein the crosslinking agent comprises a functional group A that reacts with a functional group B in molecules in the biological sample to form a covalent bond.

4. The method of claim 3, wherein the functional group A in the crosslinking agent reacts with the functional group B in molecules that are endogenous in the biological sample.

5. The method of claim 4, wherein the endogenous molecules comprise an endogenous nucleic acid, an endogenous protein, an endogenous peptide, an endogenous lipid, an endogenous carbohydrate, and/or an endogenous mineral, optionally wherein the endogenous molecules comprise extracellular matrix (ECM) components.

6. The method of any one of claims 3-5, wherein the crosslinking agent comprises a plurality of functional groups A that are amine-reactive, optionally wherein the functional group A is reactive with an α- and/or ε-amino group and not reactive with an aromatic amino group.

7. The method of any one of claims 1-6, wherein the crosslinking agent comprises an amine-to-amine crosslinking moiety, optionally wherein the crosslinking agent comprises a bis(sulfosuccinimidyl)suberate (BS3) moiety or a disuccinimidyl suberate (DSS) moiety, and optionally one or more spacers between functional groups, optionally wherein the one or more spacers comprise a polyethylene glycol (PEG).

8. The method of any one of claims 1-7, wherein the crosslinked matrix comprises molecules of the crosslinking agent covalently bound to themselves.

9. The method of any one of claims 1-8, wherein the crosslinked matrix comprises molecules of the crosslinking agent covalently bound to endogenous molecules in the biological sample.

10. The method of any one of claims 1-9, wherein the crosslinked matrix is not covalently bound to an exogenous molecule or a product or complex thereof prior to generating the RCA product in (b).

11. The method of claim 10, wherein the exogenous molecule is an exogenous primer and the product or complex of the exogenous primer comprises a primer extension product.

12. The method of claim 11, wherein the product or complex of the exogenous primer comprises a cDNA sequence reverse transcribed from an endogenous RNA sequence in the biological sample.

13. The method of claim 10, wherein the exogenous molecule is an exogenous probe that directly or indirectly binds to an endogenous analyte in the biological sample.

14. The method of claim 13, wherein the exogenous probe comprises a nucleic acid sequence that directly or indirectly binds to an endogenous nucleic acid analyte or a product thereof in the biological sample, optionally wherein the exogenous probe further comprises a nucleic acid sequence that does not bind to the endogenous nucleic acid analyte or product thereof.

15. The method of claim 14, wherein the exogenous probe is a linear, circular, or circularizable probe upon direct or indirect binding to the endogenous nucleic acid analyte or product thereof.

16. The method of claim 13, wherein the exogenous probe comprises a binding moiety that directly or indirectly binds to an endogenous non-nucleic acid analyte in the biological sample, optionally wherein the binding moiety is an antibody or epitope binding fragment thereof, and optionally wherein the exogenous probe further comprises a reporter oligonucleotide that does not bind to the endogenous non-nucleic acid analyte.

17. The method of any one of claims 13-16, wherein the biological sample is contacted with the exogenous probe prior to generating the RCA product in (b).

18. The method of any one of claims 13-17, wherein the biological sample is contacted with the exogenous probe prior to the contacting with the crosslinking agent in (a).

19. The method of any one of claims 13-18, wherein the exogenous probe is circularized prior to the contacting with the crosslinking agent in (a).

20. The method of any one of claims 1-19, wherein the biological sample is not subjected to clearing prior to the generating the RCA product in (b).

21. The method of claim 20, wherein the clearing comprises hydrophobic clearing, hydrophilic clearing, and/or hydrogel-based clearing.

22. The method of any one of claims 1-21, wherein the biological sample is not subjected to lipid digestion and/or extraction prior to the generating the RCA product in (b).

23. The method of any one of claims 1-22, wherein the biological sample is not subjected to protein digestion and/or extraction prior to the generating the RCA product in (b).

24. The method of any one of claims 1-23, wherein the RCA product is generated at a location in the biological sample.

25. The method of any one of claims 1-24, wherein the RCA product is generated from a circular or circularized DNA, a circular or circularized RNA, or a circular or circularized nucleic acid probe or probe set in the biological sample.

26. The method of claim 25, wherein the RCA product is generated from a circularized cDNA.

27. The method of claim 25, wherein the RCA product is generated from a circular or circularizable nucleic acid probe or probe set hybridized to a nucleic acid in the biological sample.

28. The method of claim 27, wherein the nucleic acid is a genomic DNA, an mRNA, a cDNA, or an exogenous nucleic acid probe.

29. The method of claim 27 or 28, wherein the circularizable nucleic acid probe or probe set comprises DNA bases and optionally one or more RNA bases at or near a ligation site in the circularizable nucleic acid probe or probe set.

30. The method of claim 29, wherein the one or more RNA bases are at a 3′ ligatable end or within five nucleotide residues from the 3′ ligatable end in the circularizable nucleic acid probe or probe set.

31. The method of any one of claims 1-30, wherein the RCA product is generated using a circular template that is covalently or noncovalently linked to an endogenous molecule in the biological sample and/or the crosslinked matrix.

32. The method of any one of claims 1-31, wherein the RCA product is generated using a circular template that hybridizes to a nucleic acid which is covalently or noncovalently linked to an endogenous molecule in the biological sample and/or the crosslinked matrix.

33. The method of any one of claims 1-32, wherein the RCA product is generated using a primer that is covalently or noncovalently linked to endogenous molecules in the biological sample and/or the crosslinked matrix.

34. The method of claim 33, wherein the biological sample is contacted with the primer prior to the contacting with the crosslinking agent in (a).

35. The method of any one of claims 1-34, wherein the RCA product does not comprise nucleic acid residues comprising the functional group B.

36. The method of any one of claims 1-35, wherein the RCA product does not comprise modified nucleic acid residues.

37. The method of any one of claims 1-36, wherein the RCA product is composed entirely of naturally occurring nucleic acid residues.

38. The method of any one of claims 1-37, wherein the crosslinked matrix is positively charged.

39. The method of any one of claims 1-37, wherein the crosslinked matrix is negatively charged.

40. The method of any one of claims 1-37, wherein the crosslinked matrix is charge neutral.

41. The method of any one of claims 1-40, wherein the diameter of the RCA product is smaller than the diameter of a reference RCA product generated in a reference biological sample without crosslinking the reference biological sample prior to RCA.

42. The method of claim 41, wherein (b) comprises generating a plurality of RCA products in the biological sample, and wherein the average diameter of the plurality of RCA products is smaller than the average diameter of reference RCA products generated in the reference biological sample without crosslinking the reference biological sample prior to RCA.

43. The method of claim 42, wherein the average diameter of the plurality of RCA products is at least about 5% smaller, at least about 10% smaller, at least about 15% smaller, at least about 20% smaller, at least about 25% smaller, or more than about 25% smaller than the average diameter of the reference RCA products generated in the reference biological sample.

44. The method of claim 42 or claim 43, wherein the average diameter of the plurality of RCA products is less than about 1.5 μm, less than about 1.4 μm, less than about 1.3 μm, less than about 1.2 μm, less than about 1.1 μm, less than about 1 μm, less than about 0.9 μm, less than about 0.8 μm, less than about 0.7 μm, less than about 0.6 μm, less than about 0.5 μm, less than about 0.4 μm, or less than about 0.3 μm.

45. The method of any one of claims 42-44, wherein the average diameter of the plurality of RCA products is about 0.5 μm smaller, about 0.4 μm smaller, about 0.3 μm smaller, about 0.2 μm smaller, about 0.1 μm smaller, or about 0.5 μm smaller than the average diameter of the reference RCA products generated in the reference biological sample.

46. The method of any one of claims 42-45, wherein the average distance between the plurality of RCA products in the biological sample is greater than the average distance between the reference RCA products generated in the reference biological sample.

47. The method of any one of claims 42-46, wherein the RCA to generate the plurality of RCA products and/or the RCA to generate the reference RCA products is performed for about 10 minutes, about 30 minutes, about 1 hour, about 2 hours, about 3 hours, about 5 hours, about 10 hours, about 15 hours, about 20 hours, or within a range between any of the aforementioned reaction times.

48. The method of any one of claims 42-47, wherein the average length of the plurality of RCA products is more than about 1 kb, more than about 5 kb, more than about 10 kb, more than about 25 kb, or more than about 50 kb.

49. The method of any one of claims 1-48, further comprising:
(c) contacting the biological sample with a detectable probe that hybridizes to the RCA product; and
(d) detecting a signal associated with the detectable probe at a location in the biological sample, thereby detecting the RCA product at the location.

50. The method of claim 49, wherein the detectable probe hybridizes to an identifier sequence in the RCA product that identifies an analyte in the biological sample.

51. The method of claim 50, wherein the identifier sequence comprises a nucleic acid analyte sequence or complement thereof.

52. The method of claim 50, wherein the identifier sequence comprises a barcode sequence or complement thereof, and wherein the barcode sequence identifies an analyte in the biological sample.

53. The method of any one of claims 49-52, wherein the detectable probe comprises a fluorescent label.

54. The method of any one of claims 419-53, wherein the detectable probe comprises a region for binding to a fluorescently labeled probe.

55. The method of any one of claims 49-54, wherein the detectable probe is selected from the group consisting of: a detectable probe comprising a 3′ or 5′ overhang upon hybridization to the RCA product; a detectable probe comprising a 3′ overhang and a 5′ overhang upon hybridization to the RCA product; a circular detectable probe; a circularizable detectable probe or probe set; a detectable probe or probe set comprising a split hybridization region configured to hybridize to a splint; and a combination thereof.

56. The method of any one of claims 49-55, wherein the signal is detected by imaging the biological sample.

57. The method of claim 56, wherein the imaging comprises fluorescent imaging.

58. A method for sample analysis, comprising:
(a) contacting a biological sample with a crosslinking agent to form a crosslinked matrix in the biological sample, wherein:
the biological sample is fixed prior to contacting the crosslinking agent, and
the crosslinking agent comprises a functional group that reacts with one or more endogenous molecules in the biological sample; and
(b) using a polymerase to generate a rolling circle amplification (RCA) product in the crosslinked matrix, wherein:
the biological sample is not cleared prior to generating the RCA product, and
the crosslinked matrix is not covalently bound to nucleic acid residues in the RCA product.

59. The method of claim 58, wherein (b) comprises generating a plurality of RCA products in the biological sample and wherein the average diameter of the plurality of RCA products is smaller than the average diameter of reference RCA products in a reference biological sample without a crosslinked matrix.

60. The method of claim 59, wherein the average diameter of the plurality of RCA products is at least about 5% smaller, at least about 10% smaller, at least about 15% smaller, at least about 20% smaller, at least about 25% smaller, or more than about 25% smaller than the average diameter of the reference RCA products in the reference biological sample.

61. The method of claim 59 or claim 60, wherein the average diameter of the plurality of RCA products is less than about 1 μm, less than about 0.9 μm, less than about 0.8 μm, less than about 0.7 μm, less than about 0.6 μm, less than about 0.5 μm, less than about 0.4 μm, or less than about 0.3 μm.

62. The method of any one of claims 59-61, wherein the average diameter of the plurality of RCA products is about 0.5 μm smaller, about 0.4 μm smaller, about 0.3 μm smaller, about 0.2 μm smaller, about 0.1 μm smaller, or about 0.5 μm smaller than the average diameter of the reference RCA products generated in the reference biological sample.

63. The method of any one of claims 58-62, wherein the RCA is performed for about 10 minutes, about 30 minutes, about 1 hour, about 2 hours, about 3 hours, about 5 hours, about 10 hours, about 15 hours, about 20 hours, or within a range between any of the aforementioned reaction times.

64. The method of any one of claims 58-63, further comprising:
(c) contacting the biological sample with a detectable probe that hybridizes to the RCA product; and
(d) detecting a signal associated with the detectable probe at a location in the biological sample, thereby detecting the RCA product at the location.

65. A method for sample analysis, comprising:
(a) providing a biological sample comprising an analyte, wherein the biological sample is a fixed biological sample;
(b) contacting the biological sample with a circularizable probe or probe set that directly or indirectly binds to the analyte in the biological sample;
(c) circularizing the circularizable probe or probe set to form a circularized probe;
(d) contacting the biological sample with a crosslinking agent to form a crosslinked matrix in the biological sample, wherein the crosslinking agent comprises a functional group that reacts with an endogenous molecule in the biological sample;
(e) using a polymerase to generate a rolling circle amplification (RCA) product in the crosslinked matrix using the circularized probe as a template;
(f) contacting the biological sample with a detectable probe that hybridizes to the RCA product; and
(g) detecting a signal associated with the detectable probe at a location in the biological sample, thereby detecting the RCA product at the location.

67. The method of claim 65 or 66, wherein the crosslinking agent does not react with the circularizable probe or probe set in the biological sample.

68. The method of any of claims 65-67, wherein the biological sample is not cleared prior to generating the RCA product.

69. The method of any one of claims 65-68, wherein the crosslinked matrix is not covalently bound to nucleic acid residues in the RCA product.

70. A method for sample analysis, comprising:
(a) providing a biological sample comprising an analyte, wherein the biological sample is a fixed biological sample;
(b) contacting the biological sample with a crosslinking agent to form a crosslinked matrix in the biological sample;
(c) contacting the biological sample with a circularizable probe or probe set that directly or indirectly binds to the analyte in the biological sample;
(d) circularizing the circularizable probe or probe set to form a circularized probe;
(e) using a polymerase to generate a rolling circle amplification (RCA) product in the biological sample using the circularized probe as a template, wherein the polymerase is optionally a phi29 polymerase;
(f) contacting the biological sample with a detectable probe that hybridizes to the RCA product; and
(g) detecting a signal associated with the detectable probe at a location in the biological sample, thereby detecting the RCA product at the location, thereby detecting the analyte in the fixed biological sample.
